(19) United States
(12) Patent Application Publication
(10) Pub. No.: US 2008/0262011 A1
(43) Pub. Date: Oct. 23, 2008

(54) Modulator

(76) Inventors: David Selwood, London (GB); Cristina Visintin, London (GB); David Baker, London (GB); Gareth Pryce, London (GB); Masahiro Okuyama, Osaka (JP)

Correspondence Address: LAHIVE & COCKFIELD, LLP;FLOOR 30, SUITE 3000, ONE POST OFFICE SQUARE, BOSTON, MA, 02109, US

(21) Appl. No.: 11/793,476

(22) PCT Filed: Dec. 21, 2005
(86) PCT No.: PCT/GB05/04990

§ 371 Date: May 1, 2008

(30) Foreign Application Priority Data
Dec. 21, 2004 (GB) 0427954.3

(51) Int. Cl. A61K 31/05; C07C 39/367; C07C 39/15; C07C 235/38; A61K 31/055; A61P 1/00; G01N 33/53; A61P 25/00; A61P 21/00; C07D 213/30; A61K 31/44; A61K 31/167

(52) U.S. Cl. 514/277; 568/746; 568/747; 568/730; 564/221; 546/344; 514/629; 514/734; 514/736; 435/7.1

(57) ABSTRACT

The present invention relates to a compound of formula I, or a pharmaceutically acceptable salt thereof. Formula (I), wherein R¹ and R² are each independently H or alkyl; Y is an alkyl group. CONR³R⁴, COOR⁵SO₂NR¹⁶R¹⁷, NHSO₂R¹⁸ or CN; X is an aryl or heteroaryl group, each of which may be optionally substituted with one or more substituents selected from (CH2)ₘZ where Z is halogen, OH, CN, alkyl, alkoxy, NO₂, CF₃, CONR⁶R⁷, CN, NR⁸R⁹, COOR¹⁰ or NHCOR¹¹ and m is 0 to 3; R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN; R¹² to R¹⁸ are each independently H or alkyl, more preferably H or Me; n is 1 to 6; wherein the compound is other than 3′,5′-dimethyl-4-(1,1-dimethylheptyl)-1,1′-biphenyl-2-ol. Further aspects of the invention related to the use of such compounds in the preparation of a medicament for the treatment of a muscular disorder, a gastrointestinal disorder, or for controlling spasticity or tremors.

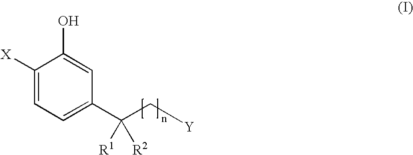

The present invention relates to compounds capable of modulating cannabinoid receptors, particularly peripheral CB₁ receptors.

### BACKGROUND TO THE INVENTION

There has recently been renewed interest in the therapeutic uses of medical cannabis and synthetic cannabinoids, such as Δ⁹-tetrahydrocannabinol (THC) [1], the active component of cannabis.

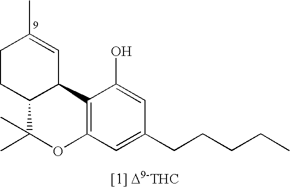

THC may be therapeutically beneficial in several major areas of medicine including control of acute and in particular chronic/neuropathic pain, nausea, anorexia, AIDS, glaucoma, asthma and in multiple sclerosis [Baker, D. et al, Nature 2000, 404, 84-87; Baker, D. et al, FASEB J 2001, 15, 300-302; Schnelle, M. et al, Forsch. Komplementarmed. 1999, 6 Suppl 3, 28-36].

A number of cannabinoid ligands have been reported in the literature. Broadly speaking, cannabinoid ligands may be divided into three main groups consisting of (i) classical cannabinoids, such as (−)-Δ⁹-tetrahydrocannabinol, Δ⁹-THC [1] and CP55,940 [9]; (ii) endocannabinoids, such as anandamide [2] and 2-arachidonoyl glycerol [3]; and (iii) non-classical heterocyclic analogues typified by heterocycles such as WIN 55,212 [7] and the selective CB₁ antagonist SR141716A [8] [Pertwee, R. G., Pharmacology & Therapeutics 1997, 74, 129-180]. Conformationally restricted anandamide analogues have also been reported [Berglund, B. A. et al, Drug Design and Discovery 2000, 16, 281-294]. To date, however, the therapeutic usefulness of cannabinoid drugs has been limited by their undesirable psychoactive properties.

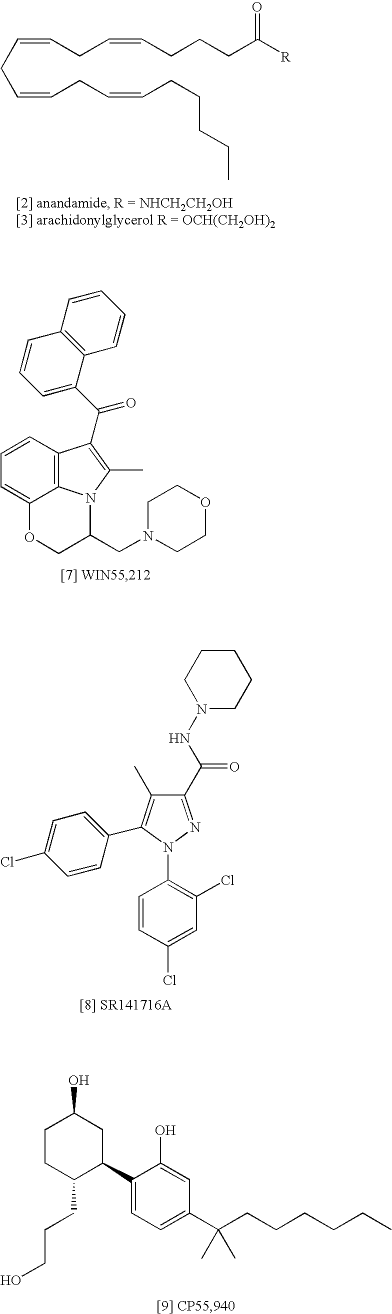

Cannabinoids are known to modulate nociceptive processing in models of acute, inflammatory and neuropathic pain [Pertwee, R. G., Prog. Neurobiol. 2001, 63, 569-611]. More specifically, research has centred on the role of cannabinoids in models of neuropathic hyperalgesia [Herzberg, U. et al, Neurosci. Lett. 1997, 221, 157-160] and inflammatory hyperalgesia [Richardson, J. D., Pain; 998, 75, 111-119; Jaggai, S. I. et al, Pain 1998, 76, 189-199; Calignano, A. et al, Nature 1998, 394, 277-281; Hanus, L. et al, Proc. Natl. Acad. Sci. U.S.A 1999, 96, 14228-14233]. It has also been suggested that cannabinoid receptor expression and the level of endogenous cannabinoids may change during inflammation and hyperalgesia [Pertwee, R. G., Prog. Neurobiol. 2001, 63, 569-611].

The cannabinoid signaling system is thought to involve two cloned cannabinoid receptors (CB₁ and CB₂), endocannabinoid ligands such as anandamide [2] and 2-arachidonoyl glycerol [3], and an endocannabinoid degradation system [Howlett, A. C. et al, International Union of Pharmacology XXVII, Pharmacol. Rev. 2002, 54, 161-202; Pertwee, R. G., Pharmacology of cannabinoid receptor ligands. Curr. Med. Chem. 1999, 6, 635-664].

One important function of the cannabinoid system is to act as a regulator of synaptic neurotransmitter release [Kreitzer, A. C. et al, Neuron 2001, 29, 717-727; Wilson, R. I. et al, Neuron 2001, 31, 453-462]. CB₁ is expressed at high levels in the CNS, notably the globus pallidus, substantia nigra, cerebellum and hippocampus [Howlett, A. C., Neurobiol. Dis. 1998, 5, 405-416]. This is consistent with the known adverse effects of cannabis on balance and short-term memory processing. [Howlett, A. C. et al, International Union of Pharmacology XXVII, Pharmacol. Rev. 2002, 54, 161-202]. CB₂ is expressed by leucocytes and its modulation does not elicit psychoactive effects; moreover it does not represent a significant target for symptom management where the majority of effects are CB₁ mediated.

Although many cannabinoid effects are centrally-mediated by receptors in the CNS [Howlett, A. C. et al, International Union of Pharmacology XXVII, Pharmacol. Rev. 2002, 54, 161-202], it is understood that peripheral CB receptors also play an important role, particularly in pain and in the gastrointestinal tract. For example, CB₁ is also expressed in peripheral tissues, such as in dorsal root ganglia, peripheral nerves and neuromuscular terminals, thereby allowing neurotransmission to be regulated outside the CNS [Pertwee, R. G., Life Sci. 1999, 65, 597-605]. Accordingly, therapeutic activity in conditions such as those involving pain [Fox, A. et al, Pain 2001, 92, 91-100] or gut hypermotility, may be located in non-CNS sites. To date, however, research into the peripheral cannabinoid system has been hampered by the lack of pharmacological agents that selectively target peripheral receptors over those of the CNS.

In order to eliminate adverse psychoactive effects, it is desirable to exclude CB₁ agonists from the CNS. There are two established methods for CNS exclusion of small molecule agents. Firstly, one method involves excluding substances from the CNS by carefully controlling their physicochemical properties so as to prevent them crossing the blood brain barrier (BBB). The BBB is formed by brain endothelial cells, with tight intercellular junctions and little fenestration [Tamai, I. et al, J. Pharm. Sci. 2000, 89, 1371-1388]. Consequently, substances must enter the brain either by passive diffusion across plasma membranes or by active transport mechanisms. The BBB thus forms an effective barrier to many peripherally circulating substances.

An alternative method of excluding compounds from the brain is to incorporate structural features which enable them to be actively pumped across the BBB. One such example is the opioid agonist loperamide; although lipophilic, loperamide contains structural features recognized by the p-glycoprotein transporter (MDR1) that allow it to be actively pumped across the blood brain barrier [Wandel, C. et al, Anesthesiology 2002, 96, 913-920; Seelig, A. et al, Eur. J. Pharm. Sci. 2000, 12, 31-40].

The present invention seeks to provide cannabinoid receptor modulators that alleviate and/or eliminate some of the disadvantages commonly associated with prior art modulators, for example undesirable psychoactive side effects. More specifically, though not exclusively, the invention seeks to provide modulators that selectively target peripheral CB₁ receptors over central CB₁ receptors.

### STATEMENT OF INVENTION

A first aspect of the invention relates to a compound of formula I, or a pharmaceutically acceptable salt thereof,

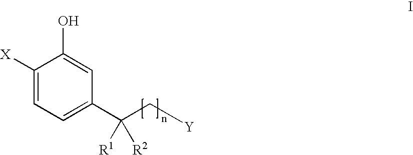

wherein

R¹ and R² are each independently H or alkyl;

Y is an alkyl group, CONR³R⁴, COOR⁵, SO₂NR¹⁶R¹⁷, NHSO₂R¹⁸ or CN;

X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;

R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;

R¹² to R¹⁸ are each independently H or alkyl;

n is 1 to 6;

wherein the compound is other than 3′,5′-dimethyl-4-(1,1-dimethylheptyl)-1,1′-biphenyl-2-ol.

Advantageously, the compounds of the present invention exhibit improved aqueous solubility and/or decreased lipophilicity compared to prior art cannabinoid receptor modulators.

A second aspect of the invention relates to a pharmaceutical composition comprising a compound of formula I as defined above admixed with a pharmaceutically acceptable diluent, excipient or carrier.

A third aspect of the invention relates to the use of a compound of formula Ia, or a pharmaceutically acceptable salt thereof,

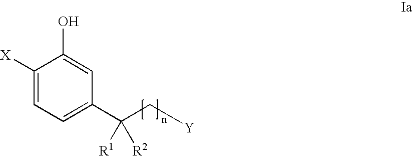

wherein

R¹ and R² are each independently H or alkyl;

Y is an alkyl group, CONR³R⁴, COOR⁵, SO₂NR¹⁶R¹⁷, NHSO₂R¹⁸ or CN;

X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;

R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;

R¹² to R¹⁸ are each independently H or alkyl;

n is 1 to 6;

in the preparation of a medicament for treating a muscular disorder.

A fourth aspect of the invention relates to the use of a compound of formula Ia as defined above, or a pharmaceutically acceptable salt thereof, in the preparation of a medicament for controlling spasticity and tremors.

A fifth aspect of the invention relates to the use of a compound of formula Ia as defined above, or a pharmaceutically acceptable salt thereof, in the preparation of a medicament for treating neuropathic pain.

A sixth aspect of the invention relates to the use of a compound of formula Ia as defined above, or a pharmaceutically acceptable salt thereof, in the preparation of a medicament for treating a gastrointestinal disorder.

A seventh aspect of the invention relates to a method of treating a disorder associated with the modulation of peripheral CB₁ receptors, said method comprising administering to a subject in need thereof, a therapeutically effective amount of a compound of formula Ia as defined above, or a pharmaceutically acceptable salt thereof.

An eighth aspect of the invention relates to the use of a therapeutically effective amount of a compound of formula Ia as defined above, or a pharmaceutically acceptable salt thereof, in the preparation of a medicament for treating a disorder associated with the modulation of peripheral CB₁ receptors.

A ninth aspect of the invention relates to a method of inhibiting peripheral CB₁ receptors in a subject, said method comprising administering to a subject a compound of formula Ia as defined above, or a pharmaceutically acceptable salt thereof.

A tenth aspect of the invention relates to the use of a compound of formula Ia as defined above, or a pharmaceutically acceptable salt thereof, as a modulator of peripheral CB₁ receptors.

An eleventh aspect of the invention relates to the use of a compound of formula Ia as defined above, or a pharmaceutically acceptable salt thereof, in an assay for identifying further compounds capable of modulating peripheral CB₁ receptors.

### DETAILED DESCRIPTION

Cannabinoid

A cannabinoid is an entity that is capable of binding to a cannabinoid receptor, in particular CB₁ and/or CB₂. Typical cannabinoids include the 30 or so derivatives of 2-(2-isopropyl-5-methylphenyl)-5-pentylresorcinol that are found in the Indian hemp, Cannabis sativa, among which are those responsible for the narcotic actions of the plant and its extracts. Examples of cannabinoids are cannabidiol, cannabinol, trans-Δ⁹-tetrahydrocannabinol, trans-Δ⁸-tetrahydrocannabinol, and Δ⁹-tetrahydro-cannabinolic acid. Other examples of cannabinoids include anandamide, methanandamide and R(+)WIN55,212.

Endocannabinoid

This term means a cannabinoid that exists naturally in the body—as opposed to an exogenously supplied cannabinoid. Endocannabinoids are discussed by Di Marzo (1998) Biochimica et Biophysica Acta vol 1392 pages 153-175 (the contents of which are incorporated herein by reference). An example of an endocannabinoid is anandamide. Teachings on this entity and anandamide amidase may be found in U.S. Pat. No. 5,874,459. This document teaches the use of anandamide amidase inhibitors as analgesic agents.

Cannabinoid Receptor

A cannabinoid receptor is any one or more of several membrane proteins that bind cannabinol and structurally similar compounds and mediate their intracellular action. Two receptors for the psychoactive ingredient of marijuana Δ⁹-tetrahydrocannabinol (THC), the CB₁ and CB₂ cannabinoid receptors, have been found (Pertwee 1997 Pharmacol Ther vol 74 129-180). Both of these receptors are seven-transmembrane-domain G-protein-coupled receptors. CB₁ receptors are found in the brain and testis. CB₂ receptors are found in the spleen and not in the brain.

For both types of receptor, arachidonoylethanolamide (anandamide) is a putative endogenous ligand and both types are negatively coupled to adenylate cyclase decreasing intracellular cyclic AMP levels. Examples of sequences for such receptors are from Mus musculus—and include: CB₁, database code CB1R_MOUSE, 473 amino acids (52.94 kDA); CB₂, database code CB2R_MOUSE, 347 amino acids (38.21 kDa). More details on CB₁ and CB₂ now follow.

Cannabinoid receptor 1 (CB₁ or CNR1)

Background teachings on CB₁ have been presented by Victor A. McKusick et al on http://www.ncbi.nlm.nih.gov/Omim. The following information concerning CB₁ has been extracted from that source.

The cannabinoids are psychoactive ingredients of marijuana, principally delta-9-tetrahydrocannabinol, as well as the synthetic analogs Matsuda et al [Nature 346: 561-564, 1990] cloned a cannabinoid receptor from a rat brain. Using a cosmid clone of the entire coding sequence of the human gene, Modi and Bonner [Abstract, Cytogenet. Cell Genet. 58: 1915 only, 1991] mapped the human CNR locus to 6q14-q15 by in situ hybridization. Gerard et al [Biochem. J. 279: 129-134, 1991] isolated a cDNA encoding a cannabinoid receptor from a human brain stem cDNA library. The deduced amino acid sequence encoded a protein of 472 residues which shared 97.3% identity with the rat cannabinoid receptor cloned by Matsuda et al [ibid, 1990]. They provided evidence for the existence of an identical cannabinoid receptor expressed in human testis. Hoehe et al [New Biologist 3: 880-885, 1991] determined the genomic localization of the CNR gene by combination of genetic linkage mapping and chromosomal in situ hybridization. Close linkage was suggested with CGA which is located at 6q21.1-q23; maximum lod=2.71 at theta=0.0. Moreover, CNR was linked to markers that define locus D6Z1, a sequence localized exclusively to centromeres of all chromosomes and enriched on chromosome 6. Ledent et al [Science 283: 401-404, 1999] investigated the function of the central cannabinoid receptor (CB₁) by disrupting the gene in mice. Mutant mice did not respond to cannabinoid drugs, demonstrating the exclusive role of CB₁ in mediating analgesia, reinforcement, hypothennia, hypolocomotion, and hypotension.

Cannabinoid Receptor 2 (CB₂ or CNR2)

Background teachings on CB₂ have been presented by Victor A. McKusick et al on http://www.ncbi.nlm.nih.gov/Omim. The following information concerning CB₂ has been extracted from that source.

In addition to its renowned psychoactive properties, marijuana, or its major active cannabinoid ingredient, delta-9-tetrahydrocannabinol, exerts analgesic, antiinflammatory, immunosuppressive, anticonvulsive, and antiemetic effects as well as the alleviation of intraocular pressure in glaucoma. The G protein-coupled cannabinoid receptor-1 (CNR1; 114610), which is expressed in brain but not in the periphery, apart from low levels in testis, does not readily account for the non-psychoactive effects of cannabinoids.

Using PCR with degenerate primers to screen a promyelocytic leukemia cell cDNA library [Munro, Nature 365: 61-65, 1993] obtained a cDNA encoding CNR2, which the authors called CX5. Sequence analysis predicted that the deduced 360-amino acid 7-transmembrane-spanning protein has 44% amino acid identity with CNR1 overall and 68% identity with the transmembrane residues proposed to confer ligand specificity. Binding analysis determined than CNR2 encodes a high-affinity receptor for cannabinoids, with higher affinity than CNR1 for cannabinol. Northern blot analysis revealed that the expression of 2.5- and 5.0-kb transcripts in the HL60 myeloid cell line increases on myeloid, or granulocyte, differentiation. Using the rat CX5 homolog, Munro [1993, ibid] found that the 2.5-kb transcript is expressed in spleen but not in brain, kidney, lung, thymus, liver, or nasal epithelium. In situ hybridization analysis demonstrated expression in splenic marginal zones. PCR analysis detected CNR2 expression in purified splenic macrophages but not in CD5+ T cells. Munro [1993, ibid] speculated that the location of CNR suggests that its endogenous ligand should have an immunomodulatory role. The International Radiation Hybrid Mapping Consortium mapped the CNR2 gene to chromosome (stSG90).

Compounds

As mentioned hereinabove, the compounds of the present invention preferably exhibit improved aqueous solubility and/or decreased lipophilicity compared to prior art cannabinoid modulators. Preferably, the compounds of the invention do not cross the blood-brain barrier to any substantial extent.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₀ alkyl group, more preferably still, a C₁₋₆ alkyl group. Particularly preferred alkyl groups include, for example, methyl, ethyl, propyl, isopropyl, butyl, isobutyl, tert-butyl, pentyl and hexyl. Preferred substituents include (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3.

As used herein, the term “aryl” refers to a C₆₋₁₀ aromatic group which may be substituted (mono- or poly-) or unsubstituted. Typical examples include phenyl and naphthyl etc. Preferred substituents include (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3.

The term “heteroaryl” refers to a substituted (mono- or poly-) or unsubstituted aryl group as defined above which contains one or more heteroatoms. Suitable heteroatoms will be apparent to those skilled in the art and include, for example, sulphur, nitrogen, oxygen, phosphorus and silicon. Preferred heteroaryl groups include pyrrole, pyrazole, pyrimidine, pyrazine, pyridine, quinoline, thiophene and furan. More preferably, the heteroaryl group is a pyridinyl group, even more preferably, a pyridin-3-yl or pyridin-4-yl group. Preferred substituents include (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3. In one preferred embodiment, Z is halogen, OH, CN, alkoxy, NO₂, CF₃, CONR⁶R⁷, CN, NR⁸R⁹, COOR¹⁰ or NHCOR¹¹.

In one particularly preferred embodiment, Z is F.

In one preferred embodiment, m is 1 to 3.

In another preferred embodiment, m is 0 or 1.

In one highly preferred embodiment, m is 0.

In one preferred embodiment, Z is selected from halo, alkyl, NHCOR¹¹ and OH.

In one preferred embodiment, R¹¹ and R¹⁵ are each independently alkyl.

In one preferred embodiment, R⁶ to R¹¹ are each: independently H or alkyl

In one particularly preferred embodiment, Z is chloro, methyl, NHCOMe or OH.

In one preferred embodiment of the invention, X is an optionally substituted phenyl group or an optionally substituted pyridyl group.

In a more preferred embodiment, X is an optionally substituted phenyl group or an optionally substituted pyridin-3-yl or pyridin-4-yl group.

In one especially preferred embodiment, X is an optionally substituted phenyl group.

In another particularly preferred embodiment, the phenyl group or pyridyl group is unsubstituted, or substituted by one or more substituents selected from hydroxy, halogen, alkyl, hydroxyalkyl and NH—CO-alkyl.

In one even more preferred embodiment, the phenyl group or pyridyl group is unsubstituted, or substituted by one or more substituents selected from hydroxy, halogen, hydroxyalkyl and NH—CO-alkyl.

In one particularly preferred embodiment, the phenyl group or pyridyl group is unsubstituted, or substituted by one or more substituents selected from hydroxy, chloro, methyl, hydroxymethyl and acetamido.

In one particularly preferred embodiment, the phenyl group or pyridyl group is unsubstituted, or substituted by one or more substituents selected from hydroxy, chloro, hydroxymethyl and acetamido.

In an even more preferred embodiment, X is selected from phenyl, 3,5-dichloro-phenyl, 3,5-dimethylphenyl, 3-hydroxyphenyl, pyridin-3-yl, pyridin-4-yl, 3-hydroxymethylphenyl and 3-acetamidophenyl.

In one preferred embodiment, Y is an alkyl group or CONR³R⁴. More preferably, Y is alkyl.

In one particularly preferred embodiment, R³ and R⁴ are each independently H or alkyl.

In one especially preferred embodiment, Y is an ethyl group or CONMe₂.

Preferably, n is 1 to 4.

Even more preferably, n is 4 or 5, more preferably 5.

In one preferred embodiment, R¹ and R² are each independently alkyl. More preferably, R¹ and R² are the same.

In a more preferred embodiment, R¹ and R² are both methyl.

In one preferred embodiment, Y is methyl and n is 5.

In another preferred embodiment, Y is CONMe₂ and n is 4.

In one especially preferred embodiment, the compound of formula I is selected from the following:

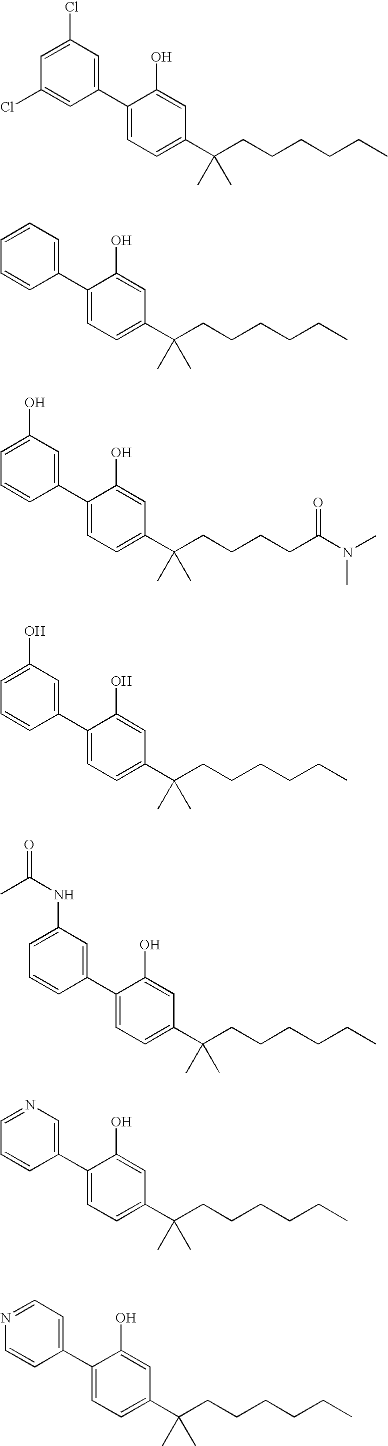

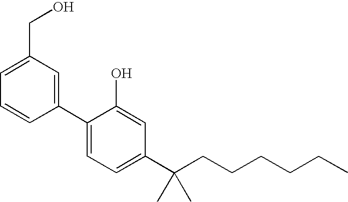

In one highly preferred embodiment of the invention, the compound of formula I is

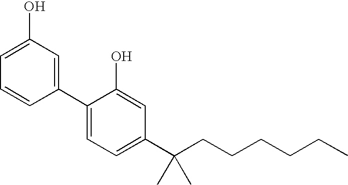

The compounds of the invention were investigated for CB₁ receptor binding and activation in vitro and for psychoactive potential in vivo, using mice. CNS levels were quantified using direct measurement of compound brain levels (for compounds lacking CNS effects). Peripheral cannabinoid activation was assessed using gut motility assays. Further details of the binding studies may be found in the accompanying Examples section.

Therapeutic Applications

Another aspect relates to the use of a compound of formula Ia as defined above, or a pharmaceutically acceptable salt thereof, in the preparation of a medicament for treating a muscular disorder.

For compounds of formula Ia, preferred embodiments are identical to those set forth above for compounds of formula I.

In one especially preferred embodiment, the compound of formula Ia is selected from the following:

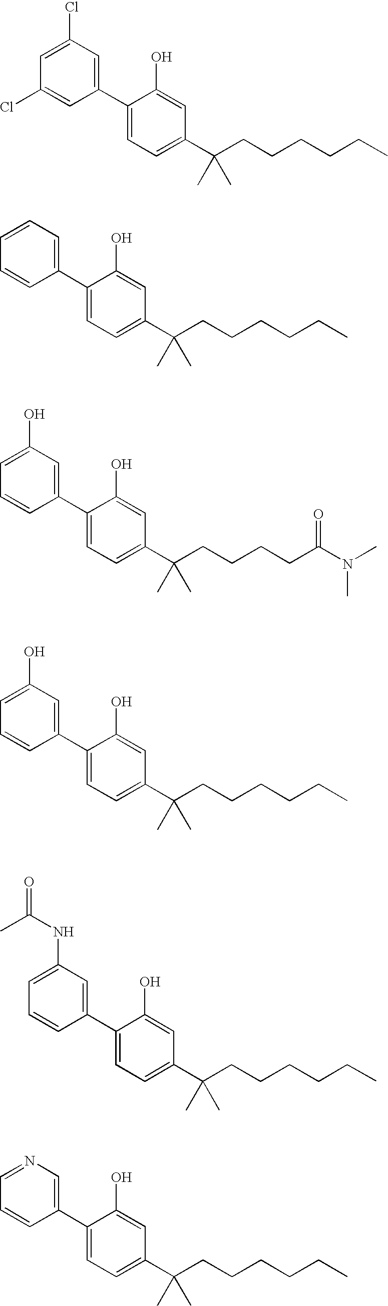

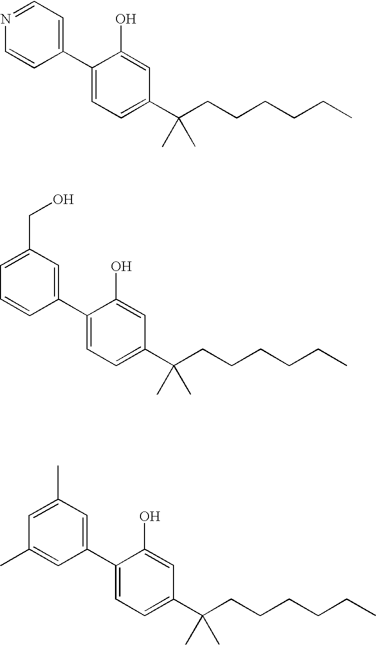

In one preferred embodiment, the muscular disorder is a neuromuscular disorder.

As used herein the phrase “preparation of a medicament” includes the use of a compound of formula Ia directly as the medicament in addition to its use in a screening programme for further agents or in any stage of the manufacture of such a medicament.

The term “muscular disorder” is used in a broad sense to cover any muscular disorder or disease, in particular a neurological disorder or disease, more particularly, a neurodegenerative disease or an adverse condition involving neuromuscular control. Thus, the term includes, for example, CREAE, MS, spasticity, Parkinson's disease, Huntingdon's Chorea, spinal cord injury, epilepsy, Tourettes' syndrome, and bladder spasm. Although there is no clear role for peripheral cannabinoid receptors in controlling spasticity in multiple sclerosis and EAE, the blood:CNS barriers are compromised in lesional areas and may provide selective access of therapeutic agents [Butter, C. et al, J. Neurol. Sci. 1991, 104, 9-12; Daniel, P. M. et al, J. Neurol. Sci. 1983, 60, 367-376; Juhler, M. et al, Brain Res. 1984, 302, 347-355].

In addition to the aforementioned disorders, the present invention also has applications in other fields where tremor or muscle spasm is present or is manifested, such as incontinence, asthma, brochial spasms, hic-coughs etc.

Another aspect relates to the use of a compound of formula Ia, or a pharmaceutically acceptable salt thereof, in the preparation of a medicament for controlling spasticity and tremors.

Yet another aspect relates to the use of a compound of formula Ia, or a pharmaceutically acceptable salt thereof, in the preparation of a medicament for treating neuropathic pain.

Compounds of formula Ia also have therapeutic applications in the treatment of various gastrointestinal disorders.

Peripheral CB₁ receptors are known to modulate gastrointestinal motility, intestinal secretion and gastroprotection. The digestive tract contains endogenous cannabinoids (anandaride and 2-arachidonoylglycerol), and cannabinoid CB₁ receptors can be found on myenteric and submucosal nerves. Activation of prejunctionally/presynaptically-located enteric (intestinal) CB₁ receptors produces inhibition of electrically-induced contractions (an effect which is associated to inhibition of acetylcholine release from enteric nerves) in various isolated intestinal tissues, including the human ileum and colon. Cannabinoid agonists inhibit intestinal motility in rodents in vivo and this effect is mediated, at least in part, by activation of peripheral (i.e. intestinal) CB₁ receptors, both in the upper gastrointestinal transit [Izzo, A. A. et al, Br. J. Pharmacol. 2000, 129, 1627-1632; Landi, M. et al, Eur. J. Pharmacol. 2002, 450, 77-83] and in the colon [Pinto, L. et al, Gastroenterology 2002, 123, 227-234]. Thus, measurement of intestinal motility, in vivo is a useful model for evaluating the activity of peripheral-acting cannabinoid drugs.

Another aspect of the invention therefore relates to the use of a compound of formula Ia, or a pharmaceutically acceptable salt thereof, in the preparation of a medicament for treating a gastrointestinal disorder.

Preferably, the gastrointestinal disorder is selected from one or more of the following: gastric ulcers, Crohn's disease, secretory diarrhea and paralytic ileus.

As used herein the term “paralytic ileus” refers to paralysis or inactivity of the intestine that prohibits the passage of material within the intestine. Typically, this may be the result of anticholinergic drugs, injury or illness. Paralytic ileus is a common occurrence post surgically.

As used herein, the term “modulator” refers to a compound or substance that increases or decreases, either directly or indirectly, the activity at a particular receptor.

Preferably for all of the above therapeutic applications, the modulator selectively modulates peripheral CB₁ receptors.

Even more preferably, the modulator selectively modulates peripheral CB₁ receptors over central CB₁ receptors.

As used herein, the term “selectively” refers to modulators that are selective for peripheral CB₁ receptors. Preferably they are selective over central CB₁ receptors. Preferably the modulators of the invention have a selectivity ratio for peripheral CB₁ receptors of greater than 10, more preferably greater than 50, more preferably greater than 100, even more preferably greater than 300 to 1, over central CB₁ receptors. Selectivity ratios may readily be determined by the skilled person.

For some applications, preferably the modulator of the present invention has a EC₅₀ value of less than about 1000 nM, preferably less than 100 nM, more preferably less than about 75 nM, even more preferably less than about 50 nM, more preferably less than about 25 nM, more preferably less than about 20 nM, more preferably less than about 15 nM, more preferably less than about 10 nM, and more preferably still, less than about 5 nM.

More preferably, the modulator binds substantially exclusively to peripheral CB₁ receptors.

In one particularly preferred embodiment, the modulator is a CB₁ receptor agonist. As used herein the term “agonist” is used in its normal sense in the art, i.e., a chemical compound which functionally activates the receptor to which it binds.

In one particularly preferred embodiment, the modulator does not substantially agonise central CB₁ receptors.

Even more preferably still, the modulator is substantially excluded from the CNS. Thus, the modulator is capable of modulating peripheral CB₁ receptors without producing CNS effects, such as undesirable psychoactive effects.

Another aspect of the invention relates to a method of treating a disorder associated with the modulation of peripheral CB₁ receptors, said method comprising administering to a subject in need thereof, a therapeutically effective amount of a compound of formula Ia as defined above.

Yet another aspect of the invention relates to the use of a therapeutically effective amount of a compound of formula Ia as defined above, or a pharmaceutically acceptable salt thereof, in the preparation of a medicament for treating a disorder associated with the modulation of peripheral CB₁ receptors.

Preferably, said disorder is associated with peripheral CB₁ receptor deactivation. More preferably, the disorder is selected from those specific conditions set forth above, for example, a muscular disorder, spasticity and tremors, neuropathic pain or a gastrointestinal disorder.

Pharmaceutical Compositions

A further aspect of the invention relates to a pharmaceutical composition comprising a compound defined above admixed with a pharmaceutically acceptable diluent, excipient or carrier.

Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients”, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Yet another aspect of the invention relates to a pharmaceutical composition comprising a pharmaceutically acceptable diluent, excipient or carrier, and a modulator of CB₁ receptors, wherein said modulator selectively modulates peripheral CB₁ receptors.

Salts/Esters

The compounds of the invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects of the present invention previously discussed, the invention includes, where appropriate all enantiomers and tautomers of compounds of formula I and Ia. The man skilled in the art will recognise compounds that possess an optical properties (one or more chiral carbon atoms) or tautomeric characteristics. The corresponding enantiomers and/or tautomers may be isolated/prepared by methods known in the art.

Stereo and Geometric Isomers

Some of the specific agents of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes solvate forms of compounds of formula I and Ia. The terms used in the claims encompass these forms.

Polymorphs

The invention furthermore relates to compounds of formula I and Ia in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes the compounds of the present invention in prodrug form. Such prodrugs are generally compounds of formula I or Ia wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient.

Combinations

In a particularly preferred embodiment, the one or more compounds of formula I or Ia are administered in combination with one or more other pharmaceutically active agents. In such cases, the compounds of the invention may be administered consecutively, simultaneously or sequentially with the one or more other pharmaceutically active agents.

Synthesis

Compounds of formula I or Ia may be synthesised in accordance with Schemes 1 and 2 set forth in the accompanying Examples section.

One aspect of the invention relates to a process (“Process 1”) for the preparation of compounds of formula Ia, wherein said process comprises the steps of:

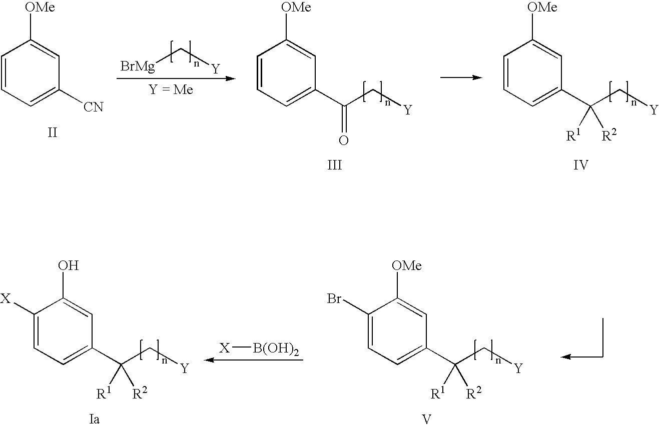

(i) reacting a compound of formula II with a compound of formula BrMg(CH₂)ₙY to form a compound of formula III;
(ii) converting said compound of formula III to a compound of formula IV;
(iii) brominating said compound of formula IV to form a compound of formula V;
(iv) converting said compound of formula V to a compound of formula I.

Preferably, R¹ and R² are both methyl.

Preferably, step (i) of Process 1 is carried out by a Grignard reaction in THF.

Preferably, step (ii) of Process 1 is carried out using TiCl₄ and a dialkyl zinc complex, for example, Me₂Zn. Preferably, the solvent is dichloromethane. Preferably, the reaction is carried out at low temperature, for example −30° C.

Preferably, step (iii) of Process 1 is carried out by treating with BBr₃, more preferably using dichloromethane as solvent, to yield the corresponding OH derivative, which is subsequently brominated to form a compound of formula V. Preferably, the bromination step is carried out using CCl₄ as solvent.

In one preferred embodiment, coupling step (iv) of Process 1 is carried out using a palladium catalyst, preferably Pd(PPh₃)₄. Typically, the reaction is carried out in the presence of a base, for example, Na₂CO₃ at elevated temperature (for example, 80° C.).

In an alternative preferred embodiment, coupling step (iv) of Process 1 is carried out using Pd(OAc)₂ and 1,4-dioxane. Typically, the reaction is carried out in the presence of dicyclohexylamine and Cs₂CO₃ at elevated temperature (for example, 80° C.).

Another aspect of the invention relates to an alternative process (“Process 2”) for preparing compounds of formula Ia, wherein said process comprises the steps of:

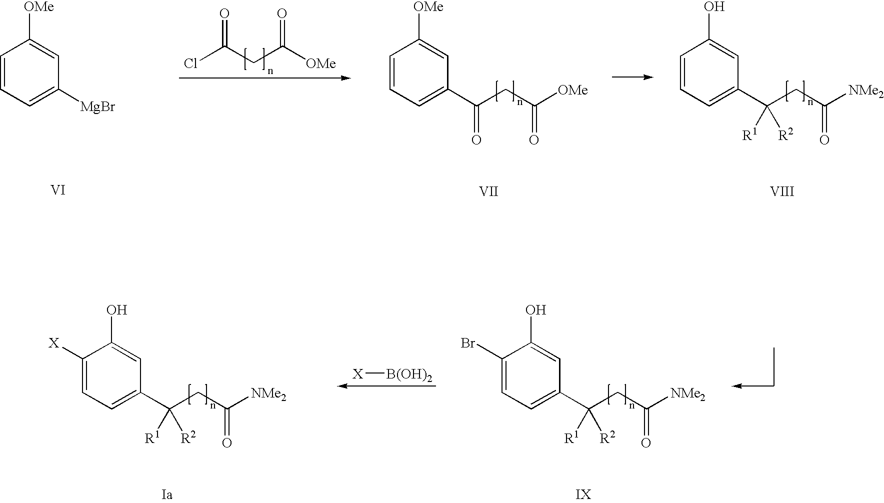

(i) reacting a compound of formula VI with a compound of formula Cl(CO)(CH₂)ₙ(CO)OMe to form a compound of formula VII;
(ii) converting said compound of formula VII to a compound of formula VIII;
(iii) brominating said compound of formula VIII to form a compound of formula IX;
(iv) converting said compound of formula IX to a compound of formula I.

Preferably, R¹ and R² are both methyl.

Preferably, step (i) of Process 2 is carried out in the presence of LiBr and CuBr. Preferably, the solvent is THF.

Preferably, step (ii) of Process 2 comprises hydrolysing the ester intermediate VII to the corresponding free acid, VIIIa.

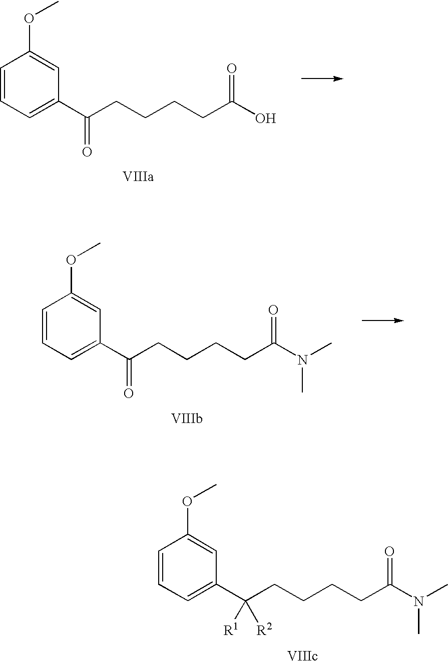

For Process 2, more preferably the hydrolysis reaction of VII is carried out under basic conditions, more preferably, using NaOH (for example 1M) and acetonitrile. Preferably, the free acid VIIIa is then converted to the corresponding amide VIIIb by treatment with, for example, Et₃N and ethyl chloroformate, followed by treatment with dimethylamine hydrochloride, H₂O and Et₃N in THF.

Preferably, amide VIIIb is then converted to a compound of formula VIIIc by treating with TiCl₄ and an alkylating agent, for example, Me₃Al. Preferably, the solvent is dichloromethane. Preferably, the reaction is carried out at low temperature, for example, −45° C.

Preferably, the compound of formula VIIIc is treated with BBr₃ in dichloromethane to form a compound of formula VIII.

Preferably, step (iii) of Process 2 is carried out by treating said compound of formula VIII with bromine in a suitable solvent, for example, CCl₄, to form a compound of formula IX

Preferably, the coupling reaction of step (iv) of Process 2 is carried out by treating said compound of formula IX with a palladium catalyst and X—B(OH)₂ in EtOH. Preferably, the catalyst is Pd(PPh₃)₄. Preferably, the coupling reaction is carried out in toluene in the presence of a base, for example, Na₂CO₃. Preferably, the reaction is carried out at elevated temperature (for example, 80° C.).

Assay

Another aspect of the invention relates to the use of a compound of formula Ia as defined hereinabove in an assay for identifying further candidate compounds that are capable of modulating one or more cannabinoid receptors.

Preferably, the cannabinoid receptor is a CB₁ receptor.

The present invention also encompasses an assay, wherein said assay is used to screen for agents or candidate compounds that can modulate peripheral CB₁ receptors. Details of such assays are presented later.

Preferably, the assay is for identifying candidate compounds that are capable of selectively modulating peripheral CB₁ receptors over central CB₁ receptors.

More preferably, the assay is a competitive binding assay.

Preferably, the candidate compound is generated by conventional SAR modification of a compound of the invention.

As used herein, the term “conventional SAR modification” refers to standard methods known in the art for varying a given compound by way of chemical derivatisation.

In one aspect, the identified compound may act as a model (for example, a template) for the development of other compounds. The compounds employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through put screen.

Techniques for high throughput drug screening may be based on the method described in Geysen, WO 84/03564. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

It is expected that the assay methods of the present invention will be suitable for both small and large-scale screening of test compounds as well as in quantitative assays.

In a preferred aspect, the assay of the present invention utilises cells that display CB₁ receptors on their surface. These cells may be isolated from a subject possessing such cells. However, preferably, the cells are prepared by transfecting cells so that upon transfection those cells display on their surface CB₁ receptors.

The above methods may be used to screen for a candidate compound useful as a modulator of one or more cannabinoid receptors.

The invention also relates to candidate compounds identified by the assay described hereinabove.

Yet another aspect of the invention relates to a pharmaceutical composition comprising one or more candidate compounds identified by the assay described hereinabove.

Another aspect of the invention relates to the use of a candidate compound identified by the method described hereinabove in the preparation of a pharmaceutical composition for use in the treatment of one or more of a muscular disorder, a gastrointestinal disorder, neuropathic pain, or for controlling spasticity and tremors.

Reporters

A wide variety of reporters may be used in the assay methods (as well as screens) of the present invention with preferred reporters providing conveniently detectable signals (eg. by spectroscopy). By way of example, a reporter gene may encode an enzyme which catalyses a reaction which alters light absorption properties.

Other protocols include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes may even be used. These and other assays are described, among other places, in Hampton R et al [1990, Serological Methods, A Laboratory Manual, APS Press, St Paul Minn.] and Maddox D E et al [1983, J Exp Med 15 8:121 1].

Examples of reporter molecules include but are not limited to (galactosidase, invertase, green fluorescent protein, luciferase, chloramphenicol, acetyltransferase, (glucuronidase, exo-glucanase and glucoamylase. Alternatively, radiolabelled or fluorescent tag-labelled nucleotides can be incorporated into nascent transcripts which are then identified when bound to oligonucleotide probes.

By way of further examples, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

Candidate Compounds

As used herein, the term “candidate compound” includes, but is not limited to, a compound which may be obtainable from or produced by any suitable source, whether natural or not.

The candidate compound may be designed or obtained from a library of compounds, which may comprise peptides, as well as other compounds, such as small organic molecules and particularly new lead compounds. By way of example, the candidate compound may be a natural substance, a biological macromolecule, or an extract made from biological materials—such as bacteria, fungi, or animal (particularly mammalian) cells or tissues, an organic or an inorganic molecule, a synthetic candidate compound, a semi-synthetic candidate compound, a structural or functional mimetic, a peptide, a peptidomimetic, a derivatised candidate compound, a peptide cleaved from a whole protein, or a peptide synthesised synthetically, for example, either using a peptide synthesiser or by recombinant techniques or combinations thereof, a recombinant candidate compound, a natural or a non-natural candidate compound, a fusion protein or equivalent thereof and mutants, derivatives or combinations thereof. The candidate compound may even be a compound that is a known modulator of CB₁ receptors that has been modified in some way eg. by recombinant DNA techniques or chemical synthesis techniques.

Typically, the candidate compound will be prepared by recombinant DNA techniques and/or chemical synthesis techniques.

Once a candidate compound capable of modulating CB₁ receptors has been identified, further steps may be carried out to select and/or to modify the candidate compounds and/or to modify existing compounds, such that they are able to modulate CB₁ receptors.

CB₁ Receptor and CB₂ Receptor Binding Assays

Details of a CB₁ receptor binding assay and a CB₂ receptor binding assay may be found in Petrocellis et al [2000 FEBS Letter 483 52-56]. The relevant Information about those assays from that reference now follows. Other assays may be used.

Displacement assays for CB₁ receptors were carried out by using ³H]SR141716A (0.4 nM, 55 Ci/mmol, Amersham) as the high affinity ligand, and the filtration technique previously described [12-14], on membrane preparations (0.4 mg/tube) from frozen male CD rat brains (Charles River Italia) and in the presence of 100 μM PMSF. Specific binding was calculated with 1 μM SR 14176A (a gift from Sanofi Recherche, France) and was 84.0%. The spleen from CD rats were used to prepare membranes (0.4 mg/tube) to carry out CB₂ binding assays by using [³H]WIN55, 212-2 (0.8 nM, 50.8 Cl/mmol, NEN-Dupont) as described previously [1,4], and again in the presence of 100 μM PMSF. Specific binding was calculated with 1 μM HU-348 (a gift from Prof. R. Mechoulam and Pharmos) and was 75.0%. In all cases, K₁ values were calculated by applying the Cheng-prusoff equation to the IC₅₀ values (obtained by GraphPad) for the displacement of the bound radioligand by increasing concentrations of the test compounds. Details on the specific references may be found in the document itself.

The present invention is further described by way of example, and with reference to the following figures wherein:

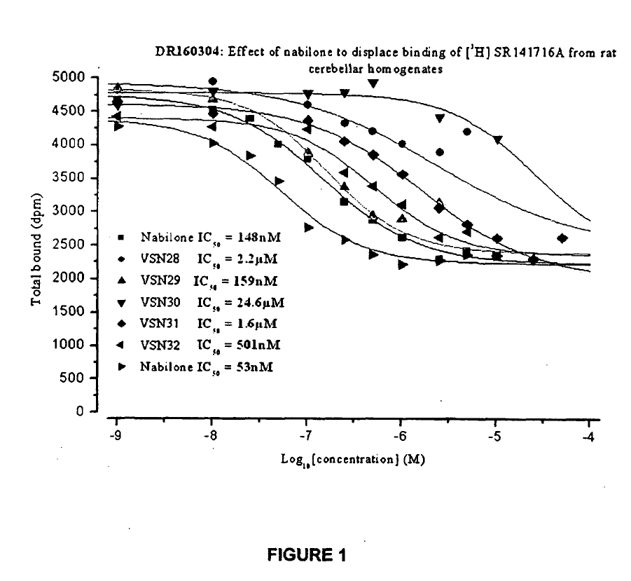

FIG. 1 shows the ability of nabilone and selected compounds of the invention to displace binding of [³H] SR141716A from rat cerebellar homogenates. The graph displays total bound (dpm) versus Log₁₀[concentration] (M).

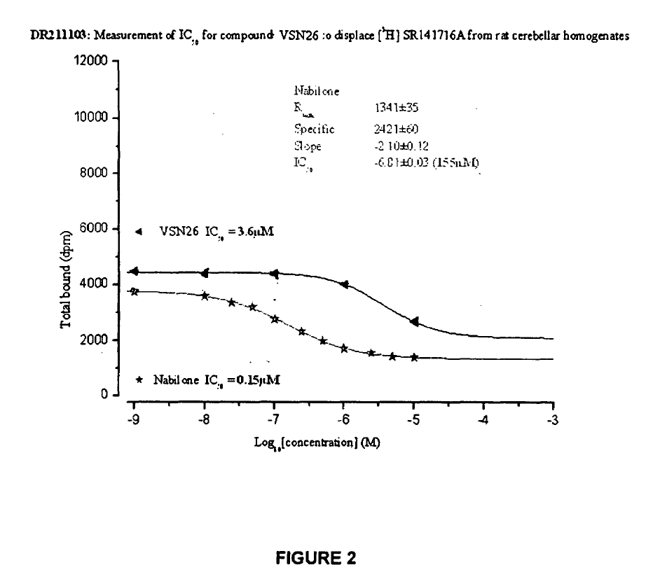

FIG. 2 shows measurement of the IC₅₀ value for compound VSN26 to displace binding of [³H] SR141716A from rat cerebellar homogenates. The graph displays total bound (dpm) versus Log₁₀[concentration] (M).

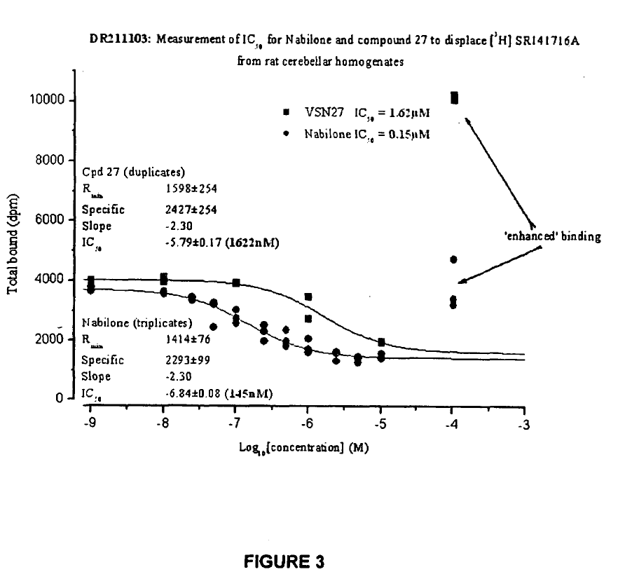

FIG. 3 shows measurement of the IC₅₀ value for compound VSN27 and nabilone to displace binding of [³H] SR141716A from rat cerebellar homogenates. The graph displays total bound (dpm) versus Log₁₀[concentration] (M).

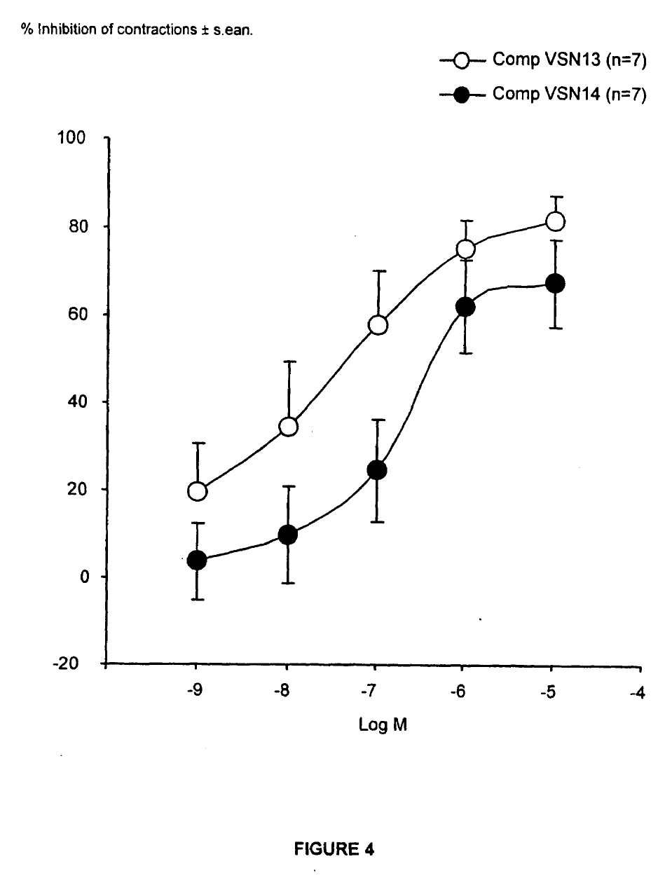

FIG. 4 shows the effect of compounds VSN13 (n=7) and VSN14 (n=7) on electrically stimulated precontracted mouse vas deferens, as described in Pertwee et al and Ward et al [Pertwee R G, Gibson T M, Stevenson L A, Ross R A, Banner W K, Saha B, Razdan R K and Martin B R (2000) O-1057, a Potent Water-Soluble Cannabinoid Receptor Agonist With Antinociceptive Properties. Br J Pharmacol 129: pp 1577-1584; Ward S, Mastriani D, Casiano F and Arnold R (1990) Pravadoline: profile in isolated tissue preparations. J Pharmacol Exp Ther 255:1230-1239]. The graph shows % inhibition of contractions against Log₁₀[concentration] (M) for compounds VSN13 and VSN14.

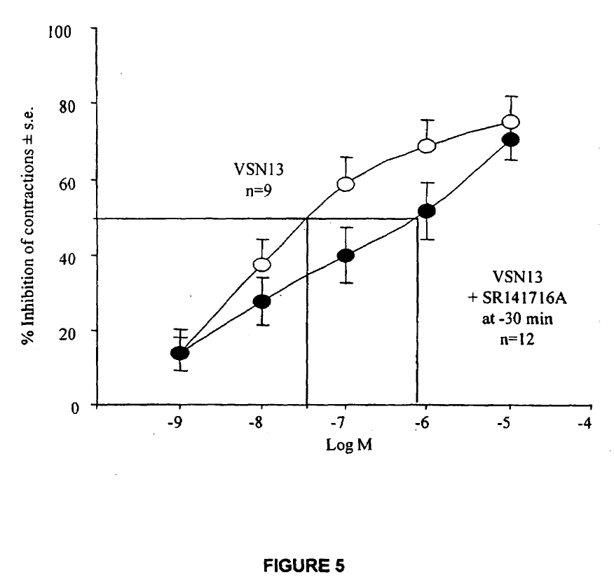

FIG. 5 shows CB₁ agonism in vitro using mouse deferens for compound VSN13. In more detail, the graph shows percentage inhibition of contractions on electrically stimulated precontracted mouse deferens as described above, versus log₁₀[concentration] (M). The graph also shows that the observed CB₁ agonism can be inhibited by the CB₁ antagonist SR141617A.

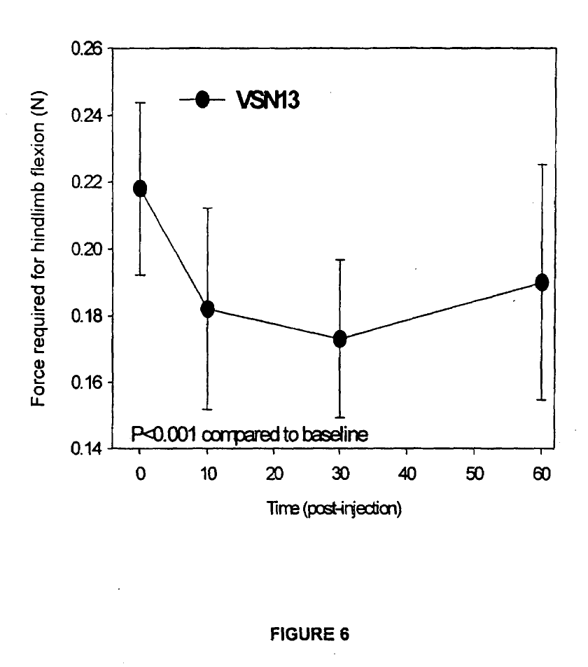

FIG. 6 shows the inhibitory effect of VSN13 in spasticity in vivo. More specifically, FIG. 6 shows the force required for hind limb flexion versus time post injection in accordance with the method described by Baker et al [Nature 2000, 404, 84-87]. VSN13 does not exhibit adverse cannabimimetic effects but is anti-spastic.

### EXAMPLES

### General

All starting materials were either commercially available or reported previously in the literature unless noted. Solvents and reagents were used without further purification except tetrahydrofuran (THF), which was dried over sodium. Reactions were monitored by thin layer chromatography (TLC) on precoated silica gel plates (Kieselgel 60 F₂₅₄, Merck). Purification was performed by flash chromatography using the method of Still,³⁶ with the columns packed with silica gel (particle size 40-63 μM, Merck), unless otherwise stated. ¹H and ¹³C NMR spectra were recorded on a Bruker AMX-300 spectrometer. Chemical shifts are reported as ppm. Coupling constants are in Hz. Mass spectra were recorded on either a VG ZAB SE spectrometer (ESP, FAB) or a Micromass Quattro electrospray liquid crystal mass spectrometer (LCMS). Some Suzuki coupling reactions were carried out using the CEM Focused Microwave™ Synthesis System.

Synthesis

Compounds of formula I were synthesised by the methods set forth below in Schemes 1 and 2.

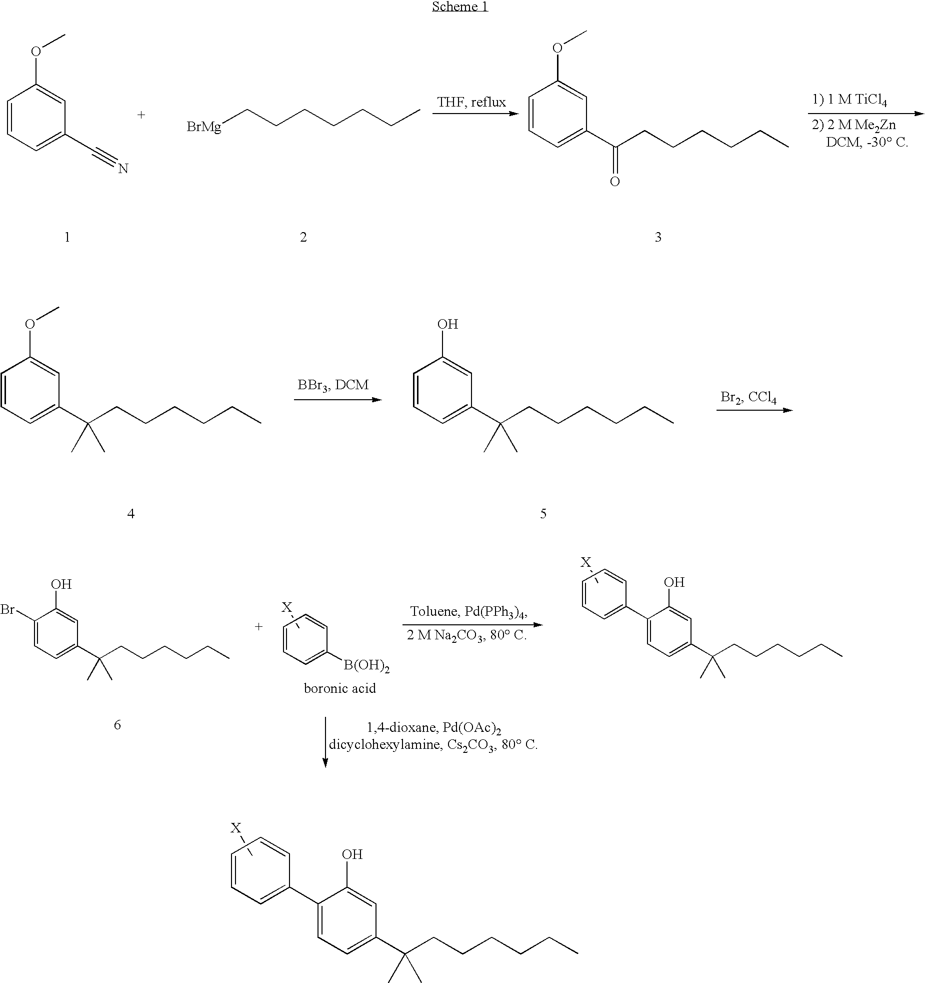

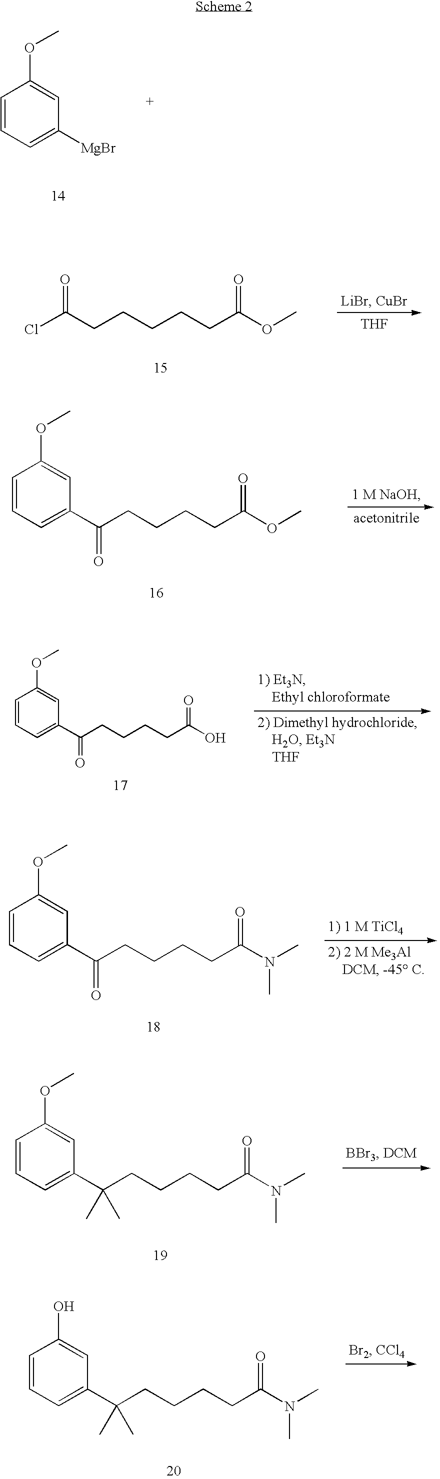

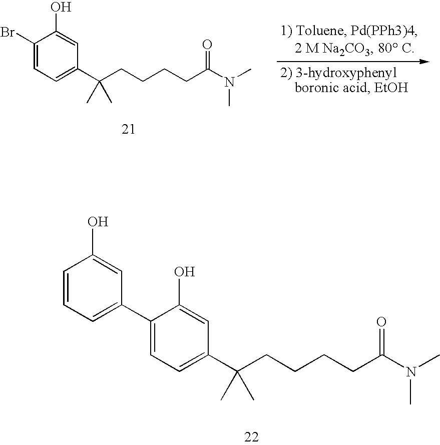

### 1-(3-Methoxyphenyl)-heptan-1-one (3)

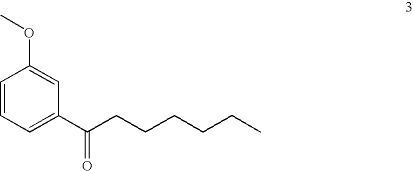

3-Methoxybenzonitrile (1) (5.33 g, 40 mmol), dry THF (100 mL), and hexylmagnesium bromide 2 M in diethyl ether (2) (30 mL, 60 mmol, 1.5 equiv.), were heated to reflux for 3 h. The reaction was cooled to 0° C. for 15 min. Hydrochloric acid (HCl) 6 M (15 mL) was added slowly, and the reaction mixture stirred and heated to reflux overnight. The solvent was removed in vacuo, the residue dissolved in ethyl acetate (EtOAc) (40 mL), and washed with HCl 6 M (20 mL). The organic layer was separated, and the aqueous layer extracted with EtOAc (3×20 mL). The combined organic layer was washed with brine (20 mL), followed by saturated aqueous sodium hydrogencarbonate (NaHCO₃) (20 mL), and then dried over magnesium sulphate (MgSO₄). The solvent was removed in vacuo and the residue chromatographed on silica eluting with 0-10% EtOAc in cyclohexane gradient. This yielded the product as a yellow oil (3) (8.93 g, 40 mmol, 100%):

¹H NMR (CDCl₃) δ 7.59-7.51 (d, 4H), 7.49-7.47 (d, J=6, 1H), 7.38-7.32 (t, 1H), 7.11-7.08 (dd, J₁=1, J₂=1, 1H), 3.85 (s, 3H), 2.96-2.91 (t, J=15, 2H), 1.74-1.67 (m, 2H), 1.41-1.25 (m, 6H), 0.90-0.86 (t, J=12, 3H);

¹³C NMR (CDCl₃) δ 200.44 (C, CO), 159.81 (C, Ar), 138.48 (C, Ar), 129.52 (CH, Ar), 120.72 (CH, Ar), 119.30 (CH, Ar), 112.32 (CH, Ar), 55.42 (CH₃), 38.76 (CH₂), 31.68 (CH₂), 29.05 (CH₂), 24.42 (CH₂), 22.55 (CH₂), 14.06 (CH₃);

MS (FAB⁺) m/z 221 (M+1).

### 1-(3-Methoxyphenyl)-3-(1,1-dimethyl)-heptane (4)

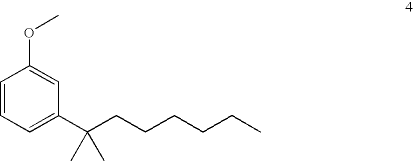

Anhydrous dichloromethane (DCM) (100 mL) was added to a 2-necked flask equipped with a thermometer and an addition funnel, and cooled to −40° C. Titanium tetrachloride 1 M in DCM (100 mL, 100 mmol) was added dropwise to the anhydrous DCM solution, and the temperature maintained between −40° C. and −30° C. After the addition, the mixture was cooled to −50° C., dimethylzinc 2 M in toluene (50 mL, 100 mmol) was added and the temperature maintained between −50° C. and −40° C. Upon addition, the orange/brown suspension was stirred for 10 min. A solution of 3 (8.95 g, 40 mmol) in dry DCM (20 mL) was added rapidly maintaining a temperature of −50° C. The mixture was stirred for 2 h a −45° C. The temperature was allowed to rise to −10° C. over 2 h with continuous stirring. The mixture was poured onto water and ice (400 mL) and the aqueous layer extracted with DCM (3×25 mL). The combined organic layer was washed with brine (50 mL), dried (MgSO₄), and concentrated to afford the crude product. The residue was chromatographed on silica eluting with a 0-5% gradient of EtOAc in cyclohexane, to yield the product as a yellow oil (4) (8.17 g, 34.9 mmol, 87%):

¹H NMR (CDCl₃) δ 7.27-7.22 (t, J=15, 1H), 6.97-6.90 (dt, 2H), 6.75-6.72 (dd, J₁=1, J₂=1, 1H), 3.76-3.75 (s, 3H), 1.63-1.57 (m, 2H), 1.30-1.27 (bs, J=9, 6H), 1.10-1.07 (bs, J=9, 6H), 0.97-0.91 (m, 2H), 0.88-0.84 (t, 3H);

¹³C NMR (CDCl₃) δ 159.38 (C, Ar), 151.71 (C, Ar), 128.85 (CH, Ar), 118.47 (CH, Ar), 112.64 (CH, Ar), 109.72 (CH, Ar), 55.11 (CH₃), 44.62 (CH₂), 31.82 (CH₂), 30.07 (CH₂), 29.00 (CH₃), 24.70 (CH₂), 22.71 (CH₂), 14.12 (CH₃);

MS (FAB⁺) m/z 234 (M+1).

### 3-(1,1-Dimethylheptyl)-phenol (5)

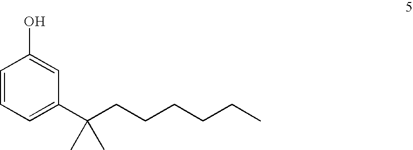

Compound 4 (4.08 g, 17.74 mmol) and boron tribromide 1 M in DCM (35 mL, 35 mmol) were stirred for 2 h. The mixture was poured onto ice and water (400 mL), and the aqueous layer extracted with DCM (30 mL), dried (MgSO₄), and the solvent removed in vacuo. The residue was purified by flash chromatography, eluting with 20% EtOAc/cyclohexane to give the product as a brown oil (5) (3.66 g, 16.63 mmol, 94%):

¹H NMR (CDCl₃) δ 7.19-7.14 (t, J=15, 1H), 6.92-6.89 (dd, J₁=1, J₂ 1, 1H), 4.83 (bs, 1H), 1.59-1.53 (m, 2H), 1.27-1.25 (d, J=6, 6H), 1.21-1.18 (d, 6H), 0.86-0.82 (t, J=12, 3H);

¹³C NMR (CDCl₃) δ 155.20 (C, Ar), 152.11 (C, Ar), 129.06 (CH, Ar), 118.49 (CH, Ar), 113.12 (CH, Ar), 112.12 (CH, Ar), 44.60 (CH₂), 31.80 (CH₂), 30.04 (CH₂), 28.93 (CH₃), 24.68 (CH₂), 22.69 (CH₂), 14.11 (CH₃);

MS (ESP⁻) m/z 219.3 (M−1).

### 3-(1,1-Dimethylheptyl)-6-bromophenol (6)

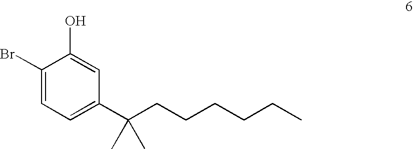

A solution of bromine (0.8575 g, 16.66 mmol) in carbon tetrachloride (CCl₄) (10 mL) was added dropwise to a solution of 5 (3.66 g, 16.66 mmol) in CCl₄ (10 mL) using an addition funnel. The mixture was stirred for a further 15 min and the temperature maintained below 30° C. The solvent was then removed in vacuo. The crude product was purified by flash chromatography, eluting with 0-10% EtOAc in cyclohexane gradient to obtain the product (6) as a viscous brown oil (3.25 g, 10.83 mmol, 65%):

¹H NMR (CDCl₃) δ 7.36-7.33 (d, J=9, 1H), 6.99-6.98 (d, 1H), 6.79-6.73 (dd, J₁=1, J₂=1, 1H), 5.41-5.40 (s, 1H), 1.57-1.51 (m, 2H), 1.24 (s, 6H), 1.16 (bs, 6H), 1.04-1.01 (m, 2H), 0.86-0.81 (t, 3H);

¹³C NMR (CDCl₃) δ 151.91 (C, Ar), 151.75 (C, Ar), 131.21 (CH, Ar), 119.76 (CH, Ar), 113.97 (CH, Ar), 106.73 (CH, Ar), 44.44 (CH₂), 31.77 (CH₂), 29.97 (CH₂), 28.87 (CH₃), 24.64 (CH₂), 22.67 (CH₂), 14.09 (CH₃);

MS (FAB⁺) m/z 300 (M+1).

### 3′,5′-Dichloro-4-(1,1-dimethylheptyl)-1,1′-biphenyl-2-ol (VSN28)

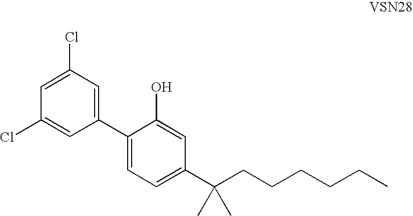

To a degassed mixture of 6 (0.10 g, 0.33 mmol) in toluene (2 mL) and aqueous 2 M sodium carbonate (Na₂CO₃) (1 mL), palladium(tetrakis)triphenylphosphine (0.0115 g, 0.033 mmol, 0.1 equiv.) was added, followed by a solution of 3,5-dichlorophenyl boronic acid (0.0699 g, 0.363 mmol, 1.1 equiv.) in ethanol (EtOH) (0.5 mL). The reaction was stirred at 80° C. for 6 h. To the reaction mixture, EtOAc (10 mL) was added, and then washed with saturated brine (10 mL), dried (MgSO₄), and concentrated to afford the crude product as a black residue. Flashtube™ chromatography was used to purify the crude product, eluting with 20% EtOAc/cyclohexane. Sections of the Flashtube™ 2008 were cut at the desired bands using the Flashtube™ Cutter (FTC). The section of silica was extracted in EtOAc, filtered off and the solvent evaporated in vacuo. The product was crystallised, and recrystallised thereafter using isopropanol and distilled water. This afforded VSB28 as brown crystals (30 mg, 0.0822 mmol, 25%):

¹H NMR (CDCl₃) δ 7.42-7.41 (d, 2H), 7.34-7.33 (d, J=3, 1H), 7.16-7.14 (d, J=6, 1H), 6.98-6.94 (dd, J=3, J₂=3, 1H), 6.89-6.88 (d, J=3, 1H), 4.89 (bs, 1H), 1.61-1.56 (m, 2H), 1.29 (s, 6H), 1.26-1.21 (d, 6H), 1.11-1.08 (m, 2H), 0.87-0.83 (t, 3H);

¹³C NMR (CDCl₃) δ 152.26 (C, Ar), 151.90 (C, Ar), 140.92 (C, Ar), 135.26 (CH, Ar), 129.70 (CH, Ar), 127.58 (CH, Ar), 127.28 (CH, Ar), 122.62 (C, Ar), 119.03 (CH, Ar), 114.04 (CH, Ar), 44.46 (CH₂), 31.76 (CH₂), 29.99 (CH₂), 28.84 (CH₃), 24.67 (CH₂), 22.64 (CH₂), 14.03 (CH₃);

MS (ESP⁻) m/z 363 (M−1).

Theoretical mass 364.13606; measured mass 364.13546.

### 3′,5′-Dimethyl-4-(1,1-dimethylheptyl)-1,1′-biphenyl-2-ol (VSN29)

[Gareau, Yves; Dufresne, Claude; Gallant, Michel; Rochette, Chantal; Sawyer, Nicole; et al.; BMCLE8; Bioorg. Med. Chem. Lett.; EN; 6; 2; 1996; 189-194]

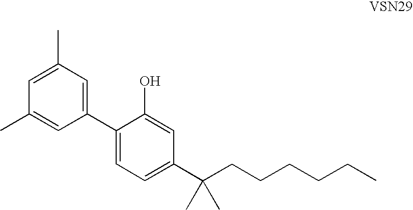

To a solution of 6 (0.100 g, 0.33 mmol) in 1,4-dioxane (2 mL) was added caesium carbonate (CsCO₃) (0.2150 g, 0.66 mmol, 2.0 equiv.). Palladium acetate (4.49 mg, 0.02 mmol) was added followed by dicyclohexylamine (8.0 μL, 0.04 mmol). Finally, 3,5-dimethylphenyl boronic acid (0.0742 g, 0.495 mmol, 1.5 equiv) was added. The sample tube was placed in the microwave (with variable power), and the reaction heated for 10 min at 80° C. DCM (1 mL) was added to the tube, and the mixture washed with 1:1 solution of saturated NaCl and saturated Na₂CO₃ (15 μL). The organic layer was dried (MgSO₄), and concentrated to give the crude product as a dark brown viscous residue. The crude product was purified by flash chromatography, eluting with 10-30% DCM in cyclohexane gradient to afford compound VSN28 as a brown viscous oil (18.2 mg, 0.0562 mmol, 17%):

¹H NMR (CDCl₃) δ 7.15-7.12 (d, J=9, 1H), 7.07 (s, 2H), 7.02 (s, 1H), 6.99 (s, 2H), 5.28 (s, 1H), 2.36 (s, 6H), 1.62-1.56 (m, 2H), 1.29 (s, 6H), 1.25-1.21 (d, 6H), 1.12-1.10 (m, 2H), 0.87-0.83 (t, 3H);

¹³C NMR (CDCl₃) δ 151.98 (C, Ar), 151.46 (C, Ar), 139.00 (C, Ar), 137.01 (C, Ar), 129.44 (CH, Ar), 129.32 (CH, Ar), 126.73 (CH), 125.06 (C, Ar), 118.35 (CH, Ar), 113.27 (CH, Ar), 44.56 (CH₂), 31.83 (CH₂), 30.08 (CH₂), 28.95 (CH₃), 24.72 (CH₂), 22.72 (CH₂), 14.12 (CH₃);

MS (ESP⁻) m/z 323.5 (M−1).

Theoretical mass 324.24530 (M+H); measured mass 324.24578.

### 4-(1,1-Dimethylheptyl)-1,1′-biphenyl-2,3′-diol (VSN13)

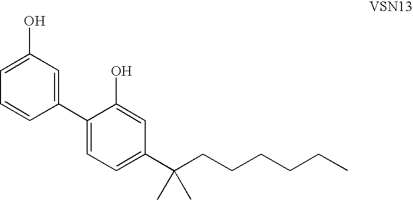

To a solution of 6 (0.100 g, 0.33 mmol) in toluene (2 mL) was added aqueous 2 M Na₂CO₃ (1 mL) and nitrogen bubbled through the mixture. Palladium(tetrakis)triphenylphosphine (0.0115 g, 0.033 mmol, 0.1 equiv.) was added and nitrogen bubbled through the mixture for an extra 5 min. Finally, 3-hydroxyphenyl boronic acid (0.0500 g, 0.363 mmol, 1.1 equiv.) in EtOH (0.5 mL) was added. The mixture was heated for 6 h at 80° C. The black slurry produced was diluted with EtOAc (10 mL) and then washed with a 1:1 solution of saturated brine and saturated Na₂CO₃ (15 mL). The aqueous layer was extracted with EtOAc (3×10 mL) and the combined organic extract dried (MgSO₄) and concentrated in vacuo to give the crude product (0.109 g). The crude residue was chromatographed using a Flashtube™ 2008, eluting with 50% EtOAc/cyclohexane to give VSN13 (35.2 mg, 0.011 mmol, 34%):

¹H NMR (CDCl₃) δ 7.36-7.33 (d, J=9, 2H), 7.23-7.17 (m, 1H), 7.05-7.02 (m, 2H), 6.79-6.76 (dd, J₁=1, J₂=3, 2H), 5.43 (bs, 1H), 5.28 (bs, 1H), 1.58-1.52 (m, 2H), 1.26-1.25 (d, 6H), 1.21-1.20 (d, 6H), 1.06-1.04 (m, 2H), 0.87-0.82 (t, 3H);

¹³C NMR (CDCl₃) δ 156.55 (C, Ar), 155.52 (C, Ar), 151.49 (C, Ar), 140.61 (C, Ar), 130.56 (CH, Ar), 129.50 (CH, Ar), 127.68 (C, Ar), 122.48 (CH, Ar), 118.95 (CH, Ar), 116.95 (CH, Ar), 114.07 (CH, Ar), 109.95 (CH, Ar), 45.00 (CH₂), 32.17 (CH₂), 30.43 (CH₂), 29.39 (CH₃), 25.09 (CH₂), 23.05 (CH₂), 14.44 (CH₃);

MS (FAB⁺) m/z 312 (M+1).

Theoretical mass 312.20892; measured mass 312.20940.

### 4-(1,1-Dimethylheptyl)-1,1′-biphenyl-2-ol (23)

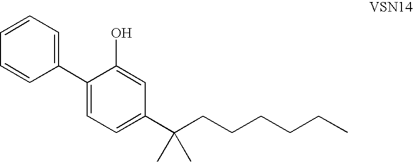

This was prepared using the same method described above for compound VSN13, using 6 (0.100 g, 0.33 mmol) and phenyl boronic acid (0.0442 g, 0.363 mmol, 1.1 equiv.). Chromatography using a Flashtube™ 2008, eluting with 50% EtOAc/cyclohexane afforded VSN14 as a light brown oil (50.2 mg, 0.17 mmol, 51%):

¹H NMR (CDCl₃) δ 7.48-7.47 (m, 2H), 7.40-7.36 (m, 1H), 7.18-7.12 (m, 1H), 6.98-6.94 (m, 2H), 5.15 (bs, 1H), 1.63-1.58 (m, 2H), 1.30 (s, 6H), 1.28-1.23 (m, 4H), 1.17-1.12 (m, 4H), 0.88-0.83 (t, J=6.4, 3H);

¹³C NMR (CDCl₃) δ 152.39 (C, Ar), 152.09 (C, Ar), 139.0 (C, Ar), 130.01 (CH, Ar), 129.59 (CH Ar), 129.45 (CH, Ar), 127.96 (CH, Ar), 118.92 (CH, Ar), 113.90 (CH, Ar), 44.92 (CH₂), 32.18 (CH₂), 30.43 (CH₂), 29.29 (CH₃), 25.09 (CH₂), 23.06 (CH₂), 14.44 (CH₃);

MS (FAB⁺) m/z 297 (M+1).

### N-[4′-(1,1-dimethylheptyl)-2′-hydroxy-1,1′-biphenyl-3-yl]-acetamide (VSN30)

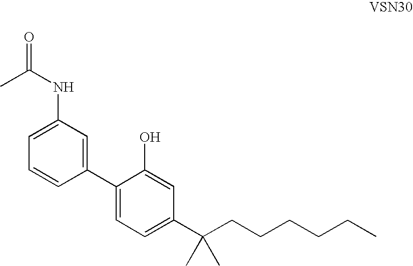

This was prepared using the same method described above for compound VSN13, using 6 (0.100 g, 0.33 mmol) and 3-acetamidophenyl boronic acid (0.0649 g, 0.363 mmol, 1.1 equiv.). Chromatography using a Flashtube™ 2008, eluting with 50% EtOAc/cyclohexane afforded VSN30 as a light brown oil (10.2 mg, 0.0290 mmol, 9%):

¹H NMR (CDCl₃) δ 7.49-7.47 (d, J=6, 2H), 7.39-7.30 (m, 2H), 7.14-7.09 (m, 2H), 6.93-6.91 (dd, J₁=3, J₂=3, 1H), 2.16-2.15 (s, 3H), 1.60-1.55 (m, 2H), 1.27-1.23 (d, 6H), 1.21-1.20 (d, 6H), 1.09-1.07 (m, 2H), 0.86-0.82 (t, J=12, 3H);

¹³C NMR (CDCl₃) δ 151.92 (C, Ar), 147.30 (C, Ar), 146.81 (C, Ar), 137.01 (C, Ar), 130.02 (CH, Ar), 129.93 (CH, Ar), 128.99 (CH, Ar), 124.32 (C, Ar), 119.90 (CH, Ar), 118.03 (CH, Ar), 113.20 (CH, Ar), 44.42 (CH₂), 31.78 (CH₂), 30.03 (CH₂), 28.89 (CH₃), 24.70 (CH₂), 22.66 (CH₂), 14.04 (CH₃).

### 5-(1,1-Dimethylheptyl)-2-pyridin-3-yl phenol (VSN26)

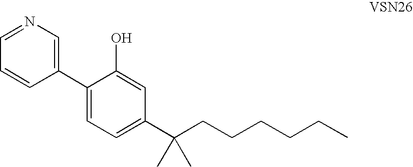

The same procedure for the synthesis of compound VSN13, was adopted, using 6 (0.100 g, 0.33 mmol) and 3-pyridine boronic acid (0.04461 g, 0.363 mmol, 1.1 equiv.). Chromatography using a Flashtube™ 2008, eluting with 50% EtOAc/cyclohexane afforded VSN26 (40.3 mg, 0.135 mmol, 41):

¹H NMR (CDCl₃) δ 8.00-7.97 (d, J=9, 1H), 7.48-7.45 (m, 2H), 7.23-7.20 (d, J=9, 2H), 7.00-6.98 (dd, J₁=3, J₂=3, 2H), 1.62-1.56 (m, 2H), 1.29 (s, 6H), 1.21 (s, 6H), 1.11-1.07 (m, 2H), 0.86-0.82 t, J=12, 3H);

¹³C NMR (CDCl₃) δ 156.75 (C, Ar), 152.43 (C, Ar), 150.71 (C, Ar), 148.05 (C, Ar), 137.07 (CH, Ar), 134.64 (CH, Ar), 130.41 (CH, Ar), 124.49 (CH, Ar), 123.21 (CH, Ar), 119.15 (CH, Ar), 109.76 (CH, Ar), 44.97 (CH₂), 32.16 (CH₂), 30.41 (CH₂), 29.38 (CH₃), 25.10 (CH₂), 23.05 (CH₂), 14.44 (CH₃);

MS (FAB⁺) m/z 298 (M+1).

Theoretical mass 298.21708 (M+H); measured mass 298.21741.

### 5-(1,1-Dimethylheptyl)-2-pyridin-4-yl phenol VSN27)

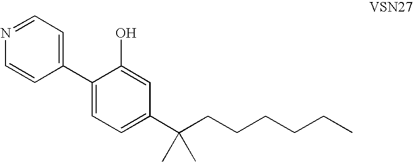

The same procedure for the synthesis of compound VSN13, was adopted, using 6 (0.100 g, 0.33 mmol) and 4-pyridine boronic acid (0.0446 g, 0.363 mmol, 1.1 equiv.). Chromatography using a Flashtube™ 2008, eluting with 50% EtOAc/cyclohexane afforded VSN27 (34.8 mg, 0.0497 mmol, 35%):

¹H NMR (CDCl₃) δ 7.83 (s, 2H), 7.71-7.69 (m, 1H), 7.58-7.55 (m, 1H), 7.30-7.27 (d, J=9, 1H), 6.87-6.85 (dd, J₁=3, J₂=3, 2H), 1.54-1.48 (m, 2H), 1.18 (s, 6H), 1.11 (s, 6H), 1.10-0.98 (m, 2H), 0.76-0.72 (t, J=12, 3H);

¹³C NMR (CDCl₃) δ 156.81 (C, Ar), 153.16 (C, Ar), 149.80 (C, Ar), 146.76 (C, Ar), 130.28 (CH, Ar), 125.07 (CH, Ar), 124.60 (CH, Ar), 119.20 (CH, Ar), 109.88 (CH, Ar), 44.93 (CH₂), 32.15 (CH₂), 30.40 (CH₂), 29.35 (CH₃), 25.09 (CH₂), 23.04 (CH₂), 14.43 (CH₃);

MS (FAB⁺) m/z 298 (M+1).

Theoretical mass 298.21708 (M+H); measured mass 298.21684.

### 4-(1,1-Dimethylheptyl)-3′-(hydroxymethyl)-1,1′-biphenyl-2-ol (VSN31)

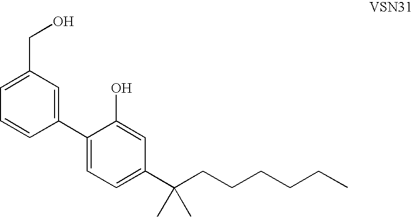

A solution of 6 (0.05 g, 0.165 mmol) was dissolved in 1,4-dioxane (2 mL), and caesium carbonate (0.1075 g, 0.33 mmol, 2.0 equiv.) added. To the resulting mixture, palladium acetate (0.0011 g, 0.01 mmol) was added followed by dicyclohexylamine (2.0 μL, 0.02 mmol). 3-Hydroxymethylphenyl boronic acid (0.0376 g, 1.5 equiv.) dissolved in MeOH (0.5 mL) was added to the mixture. The sample tube was heated in the microwave for 10 min at 80° C. The mixture was quenched with a 1:1 solution of brine and saturated Na₂CO₃ (15 mL), and the aqueous layer extracted with EtOAc (3×10 mL). The combined organic extract was dried (MgSO₄) and concentrated to give the crude product. The crude product was chromatographed in a Flashtube™ 2008 using 50% EtOAc/cyclohexane as the eluent. Chromatography yielded the pure product VSN31 (15 mg, 0.0460 mmol, 28%):

¹H NMR (CDCl₃) δ 7.49-7.39 (m, 3H), 7.29 (s, 1H), 7.18-7.15 (dd, J₁=3, J₂=3, 1H), 6.97-6.94 (dd, J₁=3, J₂=3, 2H), 4.70 (s, 2H), 1.62-1.57 (m, 2H), 1.30 (s, 6H), 1.25-1.22 (d, J=9, 6H), 1.13-1.11 (m, 2H), 0.87-0.83 (t, J=12, 3H);

¹³C NMR (CDCl₃) δ 151.93 (C, Ar), 151.76 (C, Ar), 141.85 (C, Ar), 137.55 (C, Ar), 129.63 (CH, Ar), 129.42 (CH, Ar), 128.25 (CH, Ar), 127.64 (CH, Ar), 126.13 (CH, Ar), 124.71 (C, Ar), 118.56 (CH, Ar), 113.52 (CH, Ar), 44.51 (CH₂), 31.79 (CH₂), 30.03 (CH₂), 28.90 (CH₃), 24.68 (CH₂), 22.67 (CH₂), 14.08 (CH₃).

MS (FAB⁺) m/z 326.

Theoretical mass 326.22457; measured mass 326.22575.

### Methyl-6(3-methoxyphenyl)-6-oxohexanoate (16)

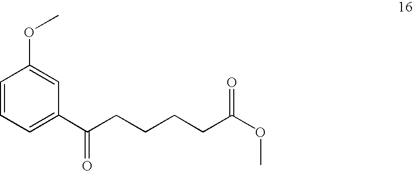

To a mixture of anhydrous lithium bromide (4.66 g, 53.74 mmol, 2.4 equiv.) and copper(I)bromide (3.805 g, 26.87 mm, ol, 1.2 equiv.) was added dry THF (50 mL) and the solution stirred at room temperature. Once homogenous, a solution of 3-methoxyphenylmagnesium bromide in THF (14) (26.87 mL, 26.87 mmol, 1.2 equiv.) was added, and soon afterwards, a solution of methyl adipoyl chloride (15) (4.0 g, 22.39 mmol) in THF (2 mL). The mixture was stirred for 30 min, quenched with saturated aqueous ammonium chloride (NH₄Cl) (20 mL), and extracted with EtOAc (3×15 mL). The organic extracts were dried (MgSO₄) and concentrated under vacuum to give the crude product as a dark green oil. The crude product was chromatographed using 10% EtOAc/cyclohexane as the eluent. Chromatography yielded the pure product (16) (3.8 g, 15.30 mmol, 68%):

¹H NMR (CDCl₃) δ 7.53-7.47 (t, 2H), 7.38-7.33 (t, J=15, 1H), 7.11-7.07 (dd, J₁=3, J₂=3, 1H), 3.85 (s, 3H), 3.66 (s, 3H), 3.01-2.97 (t, J=12, 2H), 2.39-2.34 (t, J=15, 2H), 1.77-1.68 (m, 4H);

¹³C NMR (CDCl₃) δ 199.59 (C, CO), 173.81 (C, COOMe), 159.89 (C, Ar), 138.39 (C, Ar), 129.55 (CH, Ar), 120.64 (CH, Ar), 119.41 (CH, Ar), 112.37 (CH, Ar), 55.43 (CH₃), 51.48 (CH₃), 38.23 (CH₂), 33.90 (CH₂), 24.59 (CH₂), 23.74 (CH₂).

### 6-(3-Methoxyphenyl)-6-oxohexanoic acid (17)

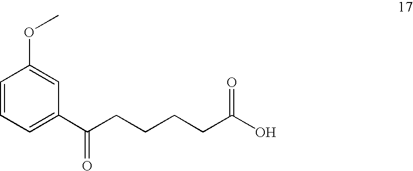

To compound 16 (1.0092 g, 0.403 mmol, was added aqueous 1 M sodium hydroxide (NaOH) (7 mL) and acetonitrile (5 mL), and the reaction stirred at room temperature overnight. The reaction mixture was washed with EtOAc, and the aqueous layer acidified with aqueous 1 M HCl. Thereafter, the aqueous layer was washed with EtOAc (3×15 mL). The organic extract was dried and concentrated to give 17 as the pure product (0.700 g, 0.296 mmol, 74%):

¹H NMR (CDCl₃) δ 7.53-7.46 (t, 2H), 7.38-7.33 (t, J=15, 1H), 7.11-7.08 (dd, J₁=3, J₂=3, 1H), 3.85 (s, 3H), 3.00-2.95 (t, J=15, 2H), 2.44-2.39 (t, J=15, 2H), 1.79-1.70 (m, 4H);

¹³C NMR (CDCl₃) δ 199.65 (C, CO), 179.23 (C, COOH), 159.89 (C, Ar), 138.34 (C, Ar) 129.57 (CH, Ar), 120.66 (CH, Ar), 119.48 (CH, Ar), 112.37 (CH, Ar), 55.44 (CH₃), 38.18 (CH₂), 33.81 (CH₂), 24.30 (CH₂), 23.63 (CH₂);

MS (FAB⁺) m/z 237 (M+1).

### 6-(3-Methoxyphenyl)-N,N-dimethyl-6-oxohexanamide (18)

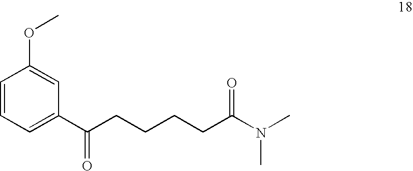

To compound 17 (0.70 g, 2.96 mmol) in dry THF (6 mL) under nitrogen atmosphere, triethylamine (0.826 mL, 5.92 mmol, 2.0 equiv.) was added and the mixture cooled to −10° C. Ethyl chloroformate (0.283 mL, 2.96 mmol, 1.0 equiv.) was, added and the reaction mixture stirred for a further 15 min at −10° C. A solution of dimethylamine hydrochloride (0.724 g, 8.88 mmol, 3.0 equiv.), distilled water (1 mL), triethylamine (2.475 mL, 17.76 mmol, 6.0 equiv.), and THF (2 mL) was prepared and added dropwise to the reaction mixture. The reaction was left to warming up to 5° C. in 1.5 h, and then stirred at room temperature for a further 30 min. The mixture was poured into a 1:1 solution of saturated brine and saturated Na₂CO₃ (50 mL), and then extracted with DCM (4×15 mL). The organic layer was dried (MgSO₄) and evaporated in vacuo, and the resulting residue purified by chromatography using 0-4% EtOH/DCM gradient to give the desired compound, 18 (0.650 g, 2.47 mmol, 83%):

¹H NMR (CDCl₃) δ 7.45-7.37 (t, 2H), 7.29-7.23 (t, 1H), 7.02-6.98 (dd, J₁=3, J₂=3, 1H), 3.75 (s, 3H), 2.91-2.84 (d, 6H), 2.30-2.25 (t, J=15, 3H), 1.70-1.61 (m, 4H);

¹³C NMR (CDCl₃) δ 199.82 (C, CO), 172.62 (C, CONMe₂), 159.79 (C, Ar), 138.36 (C, Ar), 129.50 (CH, Ar), 120.58 (CH, Ar), 119.22 (CH, Ar), 112.35 (CH, Ar), 55.34 (CH₃), 38.43 (CH₂), 37.17 (CH₃), 35.27 (CH₃), 33.07 (CH₂), 24.74 (CH₂), 24.04 (CH₂);

MS (FAB⁺) m/z 264 (M+1).

### 6-(3-Methoxyphenyl)-N,N,6-trimethylheptanamide (19)

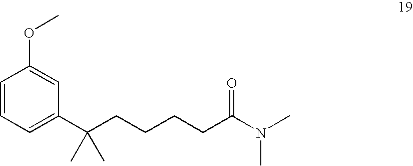

Dry DCM (4 mL) was cooled to −30° C. Titanium chloride 1 M in DCM (1.52 mL, 1.52 mmol, 1.0 equiv) was added followed by trimethylaluminum 2 M in hexane (1.52 mL, 3.04 mmol, 2.0 equiv.), and the mixture stirred for 20 min at −45° C. Compound 18 (0.400 g, 1.52 mmol) was dissolved in dry DCM (4 mL) and added dropwise to the mixture, which was allowed to warm to room temperature, and left to stir overnight. The reaction mixture was quenched with water, which was then extracted with EtOAc (3×20 mL). The organic extract was dried (MgSO₄) and concentrated to give 19 as the desired product (0.3616 g, 1.30 mmol, 85%):

¹H NMR (CDCl₃) δ 7.13-7.08 (t, J=15, 1H), 6.84-6.78 (t, 2H), 6.62-6.59 (dd, J₁=1.5, J₂=3, 1H), 3.69 (s, 3H), 2.83-2.80 (d, J=9, 6H), 2.15-2.10 (t, 2H), 1.56-1.43 (m, 4H), 1.19 (s, 6H), 1.06-1.03 (m, 2H);

¹³C NMR (CDCl₃) δ 172.91 (C, CONMe₂), 159.40 (C, Ar), 151.24 (C, Ar), 128.83 (CH, Ar), 118.28 (CH, Ar), 112.45 (CH, Ar), 109.86 (CH, Ar), 54.98 (CH₃), 44.27 (CH₂), 37.11 (CH₃), 35.17 (CH₃), 33.21 (CH₂), 28.90 (CH₃), 25.75 (CH₂), 24.66 (CH₂).

### 6-(3-Hydroxyphenyl)-N,N,6-trimethylheptanamide (20)

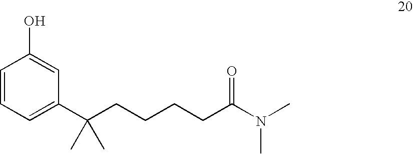

Boron tribromide 1 M in DCM (1.44 mL, 1.44 mmol, 2.0 equiv.) was added dropwise to compound 19 (0.200 g, 0.721 mmol) and the mixture stirred at room temperature for 2 h under nitrogen atmosphere. The reaction mixture was poured onto ice and water (100 mL) and the aqueous layer extracted with DCM (3×20 mL). The organic extract was dried (MgSO₄) and concentrated to give the product (20) (0.175 g, 0.66 mmol, 91%):

¹H NMR (CDCl₃) δ 7.14-7.08 (t, 1H), 6.86-6.80 (t, 2H), 6.70-6.66 (dd, J=3, 3, 1H), 2.94-2.92 (d, J=6, 6H), 2.26-2.21 (t, 2H), 1.60-1.50 (m, 4H), 1.23 (s, 6H), 1.17-1.08 (m, 2H);

¹³C NMR (CDCl₃) δ 173.77 (C, CONMe₂), 156.41 (C, Ar), 151.25 (C, Ar), 128.89 (CH, Ar), 117.47 (CH, Ar), 113.35 (CH, Ar), 112.51 (CH, Ar), 44.22 (CH₂), 37.61 (CH₃), 35.57 (CH₃), 33.36 (CH₂), 28.99 (CH₃), 25.82 (CH₂), 24.68 (CH₂);

MS (FAB⁺) m/z 264 (M+1).

### 6-(4-Bromo-3-hydroxyphenyl)-N,N,6-trimethylheptanamide (21)

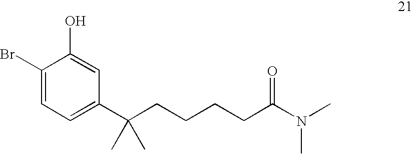

To a solution of 20 (0.050 g, 0.19 mmol) in CCl₄ (1 mL) and dry DCM (1.5 mL) was added bromine (9.7 μL, 0.19 mmol, 1.0 equiv.). The reaction mixture was stirred for 15 min below 30° C. The solvent was evaporated to give the crude residue. Chromatography using % EtOH/DCM as the eluent afforded the pure product 21 (41.7 mg, 0.122 mmol, 64%):

¹H NMR (CDCl₃) δ 7.33-7.30 (d, J=9, 1H), 7.15-7.14 (d, J=3, 1H), 6.73-6.69 (dd, J=3, 3, 1H), 2.95-2.91 (d, J=12, 6H), 2.28-2.22 (t, 2H), 1.62-1.56 (m, 4H), 1.23 (s, 6H), 1.13-1.04 (m, 2H);

¹³C NMR (CDCl₃) δ 177.42 (C, CONMe₂), 152.52 (C, Ar), 150.65 (C, Ar), 131.87 (CH, Ar), 119.37 (CH, Ar), 114.53 (CH, Ar), 106.72 (CH, Ar), 43.25 (CH₂), 37.68 (CH₃), 35.48 (CH₃), 31.61 (CH₂), 29.05 (CH₃), 26.30 (CH₂), 24.57 (CH₂);

MS (ESP⁻) m/z 340 (M−1).

### 4-(2,3′-Dihydroxy-1,1′-biphenyl-4-yl)-N,N,6-trimethylheptanamide (VSN32)

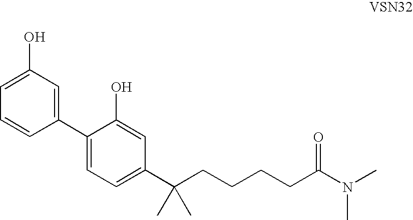

Compound 21 (0.040 g, 0.120 mmol) was dissolved in toluene (2 mL), aqueous 2 M Na₂CO₃ was added (1 mL), and nitrogen was bubbled through the mixture. Palladium(tetrakis)triphenylphosphine (0.0041 g, 0.012 mmol, 0.1 equiv.) was added, and more nitrogen bubbled through the mixture for 5 min. Finally, 3-hydroxyphenyl boronic acid (0.0182 g, 0.132 mmol, 1.1 equiv.) in EtOH (1 mL) was added to the reaction mixture. The reaction was stirred for 5 h at 80° C., after which it was quenched with a 1:1 solution of saturated brine and saturated Na₂CO₃ (15 mL). Toluene (10 mL) was added to the reaction mixture, and the separated aqueous layer washed with EtOAc (3×15 mL). The organic layer was dried (MgSO₄) and concentrated to give the crude product (64.8 mg). Chromatography using 0-2% EtOH in DCM gradient yielded the pure product VSN32 (2.45 mg, 0.0069 mmol, 6%):

¹H NMR (CDCl₃) δ 7.35-7.25 (m, 2H), 7.17-7.14 (d, J=9, 1H), 7.03-7.00 (dd, J=3, 3, 1H), 6.96-6.94 (t, 2H), 6.86-6.83 (dd, J=3, 3, 1H), 2.96-2.92 (d, J=12, 6H), 2.28-2.23 (t, 2H), 1.61-1.55 (m, 4H), 1.29 (s, 6H), 1.16-1.08 (m, 2H);

MS (ESP⁺) m/z 356.3 (M+1).

### Alternative synthesis of 4-(2,3′-Dihydroxy-1,1-biphenyl-4-yl)-N,N,6-trimethyl-heptanamide VSN32)

A screw-cap test tube containing a magnetic stir bar was charged with 6-(4-bromo-3-hydroxyphenyl)-N,N,6-trimethylheptanamide (21) (0.100 g, 0.43 mmol), Pd(OAc)₂ (0.66 mg, 2 mol %) 1 2(2′6′dimethoxybiphenyl) di cyclohexyl phosphine (2.4 mg, 2 mol %) and the 3-hydroxyphenyl boronic acid (0.059 g, 0.42 mmol) and K₂PO₃ (0.127 g, 0.66 mmol). The tube was sealed with a teflon-coated screw-cap and evacuated and backfiled with argon three times. Than dry toluene (0.6 mL) and degassed water (0.060 mL) were added and the teflon-coated screw-cap quickly replaced. The reaction mixture was vigorously stirred at 1000° C. for 1 h. The reaction mixture was then diluted with ethyl acetate (2 mL) and filtered though a thin pad of celite and concentrated under reduced pressure. Chromatography using 0-2% EtOH in DCM gradient yielded the pure product VSN32 (0.058 g, 0.16 mmol, 23.7%):

¹H NMR (CDCl₃) δ 7.35-7.25 (m, 2H), 7.17-7.14 (d, J=9, 11H), 7.03-7.00 (dd, J=3, 3, 1H), 6.96-6.94 (t, 2H), 6.86-6.83 (dd, J=3, 3, 11H), 2.96-2.92 (d, J=12, 6H), 2.28-2.23 (t, 2H), 1.61-1.55 (m, 4H), 1.29 (s, 6H), 1.16-1.08 (m, 2H);

MS (ESP⁺) m/z 356.3 (M+1).

Binding Assay

Radioligand binding assays provide a simple measure of activity at the CB₁ receptors. For functional activity, the vas deferens or cyclic 3′,5′-adenosine monophosphate (cAMP) assays described below are used. The assays are carried out with a protocol modified from the literature using the CB₁ receptor antagonist [³H] SR141716A (0.5 nM) in rat brain membranes. Male Wistar rats (150-200 g), which were allowed free access to food and water, were used. Briefly, animals were killed by cervical dislocation and cerebella dissected into ice-cold 0.25 M sucrose. Homogenates were prepared by suspension of cerebella in ice-cold 50 mM, pH 7.4 HEPES (assay) buffer using an Ultra-Turrax™ homogeniser. Homogenates were subsequently centrifuged at 45,000 rpm for 15 min at 4° C., and re-suspended in fresh ice-cold assay buffer to give a final concentration of 1 mg/mL wet weight. Compounds under test were dissolved in dimethyl sulfoxide (DMSO) and transferred to 14 mL polypropylene test tubes. [³H] SR141617A {final concentration of approximately 0.5 nM prepared in assay buffer containing 0.1% v/v tris(2-butoxyethyl)phosphate} and cerebellar membranes (400 μg wet weight per tube) were thoroughly mixed in a final volume of 0.5 mL and incubated for 1 h at 37° C. The tubes were transferred to a Brandel filtration apparatus, 5 mL ice-cold assay buffer added, and the tube contents vacuum-aspirated through Whatman GF/C glass fibre filters. A further 5 mL of ice-cold wash buffer was added to tubes and the wash/vacuum cycle repeated a further three times. The filters were transferred to plastic Mini-PolyQ™ vials, Picofluor™ liquid scintillant added, and radioactivity (dpm) was quantified using a Beckman LS6000 Liquid Scintillation Counter. The concentrations of competing ligand (test compounds) to produce 50% displacement of the radioligand (IC₅₀) from specific binding sites were computed using Origin™, by fitting total bound ligand (y-axis as dpm) against compound concentration (x-axis, log 10[x] as M) to the logistic equation y=a+b/[1+exp{−c(x−IC50)}]. Where a=lower asymptote, b=specific bound and c=slope function.

Structures and Biological Data

Table 1 below shows IC₅₀ and Log BB values for selected compounds of the invention.

Log BB (Feher M, Sourial E and Schmidt J M (2000); A Simple Model for the Prediction of Blood-Brain Partitioning. Int J Pharm 201: pp 239-247) is an empirically derived equation that relates a compounds' physicochemical characteristics to its ability to cross the blood brain barrier. Various methods have been used to calculate the penetration of the BBB; this is one of the best predictive methods.

The equation is:

log BB=log(C_{brain}/C_{blood})=0.4275−0.3873 n_{acc,solv}+0.1092 log P−0.0017 Aₚₒₗ

(n=61, r²=0.730, q²=0.688, rmse=0.424, F=51, P<0.001)

n_{acc,solv}: the number of hydrogen-bond acceptors

log P: calculated octanol-water partition coefficient

Aₚₒₗ: the polar surface area

TABLE 1

| Structure | IC₅₀ | Log BB |
|---|---|---|
| 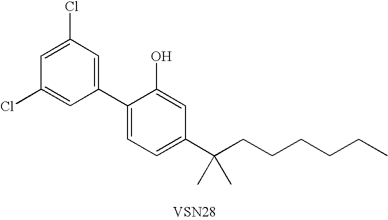 | 2.2 μM | 1.13 |
| 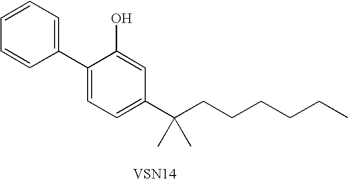 | Xtested in the vasdeferens assay | 0.81 |
| 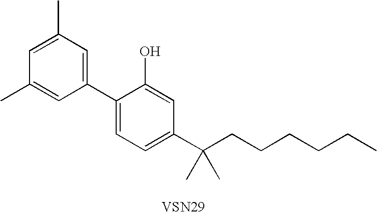 | 0.2 μM | 0.87 |
| 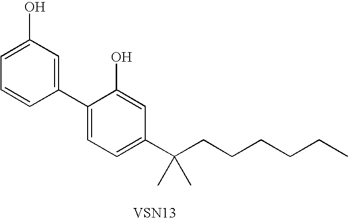 | 1.5 μMtested in the vasdeferens assay | 0.25 |
| 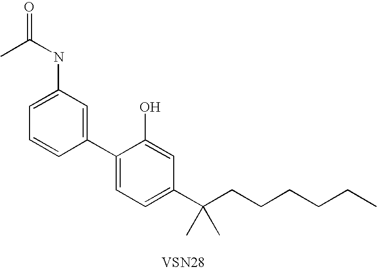 | 24.6 μM | −0.06 |
| 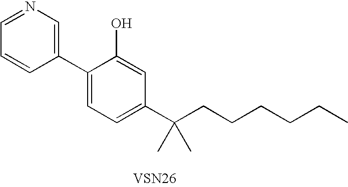 | 3.6 μM | 0.25 |
| 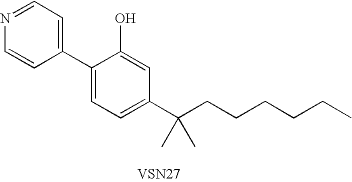 | 1.6 μM | 0.25 |
| 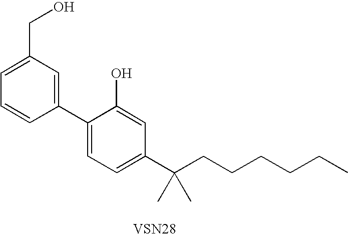 | 1.6 μM | 0.23 |
| 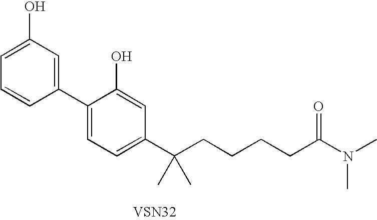 | 0.5 μM | −0.60 |

Neuropathic Pain

Hyperalgesia was assessed in accordance with the model described in WO03/066603.

Validation as CB1 Agonists with Peripheral Action

In Vitro Radioligand Binding Studies

Radioligand binding assays [Ross, R. A. et al, Br. J. Pharmacol. 1999, 128, 735-743] are carried out with the CB₁ receptor antagonist [3H]SR141716A (0.5 nM) or [3H]CP55940 (0.5 nM) in brain and spleen membranes. Assays are performed in assay buffer containing 1 mg/mL BSA, the total assay volume being 500 μL. Binding is initiated by the addition of membranes (100 μg). The vehicle concentration of 0.1% DMSO is kept constant throughout. Assays are carried out at 37° C. for 60 minutes before termination by addition of ice-cold wash buffer (50 mM Tris buffer, 1 mg/mL BSA) and vacuum filtration using a 12-well sampling manifold (Brandel Cell Harvester) and Whatman GF/B glass-fibre filters that had been soaked in wash buffer at 4° C. for 24 hours. Each reaction tube is washed five times with a 4-mL aliquot of buffer. The filters are oven-dried for 60 minutes and then placed in 5 mL of scintillation fluid (Ultima Gold XR, Packard), and radioactivity quantitated by liquid scintillation spectrometry. Specific binding is defined as the difference between the binding that occurred in the presence and absence of 1 μM unlabelled ligand and is 71% and 40% of the total radio-ligand bound in brain and spleen respectively. The concentrations of competing ligands (test compounds) to produce 50% displacement of the radioligand (IC50) from specific binding sites is calculated using GraphPad Prism (GraphPad Software, San Diego). Inhibition constant (Ki) values are calculated using the equation of Cheng & Prusoff [Cheng, Y. and Prusoff, W. H., Biochem. Pharmacol. 1973, 22, 3099-3108].

In Vitro Cannabinoid Receptor Modulating Activity

Compounds are evaluated for cannabinoid modulation potential using a mouse vas deferens preparation [Ward S, Mastriani D, Casiano F and Arnold R (1990) J Pharmacol Exp Ther 255:1230-1239] which provides evidence for CB agonism, rather than simple receptor binding which does not always reflect agonist potential.

The results for compound VSN13 are shown in FIG. 5. In more detail, the graph shows percentage inhibition of contractions on electrically stimulated precontracted mouse deferens as described above, versus log₁₀[concentration] (M). The graph also shows that the observed CB₁ agonism can be inhibited by the CB₁ antagonist SR141617A.

In Vivo Peripheral CB₁ Receptor Activation

Upper Gastrointestinal Transit

Gastrointestinal transit is measured using the charcoal method. Mice receive orally 0.1 mL (10 g/mouse) of a black marker (10% charcoal suspension in 5% gum arabic), and after 20 minutes the mice are killed by asphyxiation with CO₂ and the small intestine removed. The distance traveled by the marker is measured and expressed as a percentage of the total length of the small intestine from pylorus to caecum [Izzo, A. A. et al, Br. J. Pharmacol. 2000, 129, 1627-1632]. Cannabinoid agonists are given 30 min before charcoal administration.

Colonic Propulsion Test

Distal colonic propulsion is measured according to Pinto et al [Gastroenterology 2002, 123, 227-234]. Thirty minutes after the administration of cannabinoid drugs, a single 3 mm glass bead is inserted 2 cm into the distal colon of each mouse. The time required for expulsion of the glass bead was determined for each animal. The higher mean expulsion time value is an index of a stronger inhibition of colonic propulsion.

Psychotropic Activity of Peripherally Active Cannabinoids

Many CB₁ agonists are known to induce psychotropic associated “tetrad effects” due to central binding to CB receptors [Howlett, A. C. et al, International Union of Pharmacology. XXVII, Pharmacol. Rev. 2002, 54, 161-202]. Studies were undertaken to investigate whether the compounds of the present invention also bound to central CB₁ receptors. This is assessed by measuring the ability of the compounds to induce sedation, ptosis, hypomotility, catalepsy and hypothermia in normal mice [Brooks, J. W. et al, Eur. J. Pharmacol. 2002, 439, 83-92], following i.v., i.p. and oral administration.

Preliminary Characterization of the Biology of CB₁ Agonism

Nociceptive Activity of Peripherally Active Cannabinoids

There is evidence for CB₁ mediated nociception in the periphery [Fox, A. et al, Pain 2001, 92, 91-100]. Studies on partial sciatic nerve ligation were therefore undertaken in rats and knockout mice.

Assessment of Spasticity

Further studies were undertaken using cannabinoid knockout mice, including CB₁, CB₂, VR-1, FAAH and conditional CB₁ knockout mice. Spasticity may be induced in ABH (significant spasticity occurs in 50-60% of animals in 80 days after 3-4 disease episodes¹) or ABH.CB₁ −/− (significant spasticity occurs in 80-100% of animals in 30-40 days after 1-2 disease episodes). Compounds are injected initially intravenously (to limit first pass effects), i.p. or orally. Spasticity is assessed (n=6-7/group) by resistance to hindlimb flexion using a strain gauge [Baker, D. et al, Nature 2000, 404, 84-87]. Animals serve as their own controls and are analysed in a pairwise fashion. To reduce the number of animals, effort and expense, following a drug-free period (spasticity returns within 24 h) these animals receive different doses and or vehicle. Low doses of CB₁ agonists and CNS active CP55,940, as control, are locally (subcutaneous, intra-muscularly) administered into spastic ABH mice and the lack of activity in a contralateral limb analysed [Fox, A. et al, Pain 2001, 92, 91-100]. Expression of CB₁ in the peripheral nervous system, including dorsal root ganglia, a non-CNS site for CB-mediated nociception can be removed using peripherin-Cre transgenic mouse [Zhou, L. et al, FEBS Lett. 2002, 523, 68-72]. These conditional KO mice are maintained on the C57BL/6 background. These mice develop EAE following induction with myelin oligodendrocyte glycoprotein residues 35-55 peptide [Amor, S. et al, J. Immunol. 1994, 153, 4349-4356].

The results for compound VSN13 are shown in FIG. 6. More specifically, FIG. 6 shows the force required for hind limb flexion versus time post injection in accordance with the method described by Baker et al [Nature 2000, 404, 84-87].

In Vivo Evaluation in Normal and CREAE Mice

A CNS excluded compound provides a tool for examining if a component of a cannabinoid anti-spastic effect is mediated via peripheral CB receptors. As stated above there is no established role for peripheral cannabinoid receptors in the control of spasticity, however, spasticity is likely to be a product of nerve damage in the spinal cord, at least in EAE, [Baker, D. et al, FASEB J 2001, 15, 300-302; Baker, D. et al, J. Neuroimmunol. 1990, 28, 261-270] and aberrant signals to and from the musculature are likely, at least in part to contribute to the muscle spasms occurring in spasticity.

Various modifications and variations of the described methods of the invention will be apparent to those skilled in the art without departing from the scope and spirit of the invention. Although the invention has been described in connection with specific preferred embodiments, various modifications of the described modes for carrying out the invention which are obvious to those skilled in chemistry or related fields are intended to be within the scope of the following claims.

1. A compound of formula I, or a pharmaceutically acceptable salt thereof, 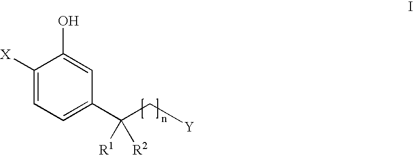
wherein
R¹ and R² are each independently H or alkyl;
Y is an alkyl group, CONR³R⁴, COOR⁵SO₂NR¹⁶R¹⁷, NHSO₂R¹⁸ or CN;
X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;
R³ to R¹¹, are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;
R¹² to R¹⁸ are each independently H or alkyl;
n is 1 to 6;
wherein the compound is other than 3′,5′-dimethyl-4-(1,1-dimethylheptyl)-1,1′-biphenyl-2-ol.

2. A compound according to claim 1 wherein X is an optionally substituted phenyl group or an optionally substituted pyridyl group.

3. A compound according to claim 1 wherein X is an optionally substituted phenyl group or an optionally substituted pyridin-3-yl or pyridin-4-yl group.

4. A compound according to claim 1 wherein m is 0 or 1

5. A compound according to claim 1 wherein Z is selected from halo, alkyl, NHCOR¹¹ and OH.

6. A compound according to claim 1 wherein R¹¹ and R¹⁵ are each independently alkyl.

7. A compound according to claim 1 wherein R⁶ to R¹¹ are each independently H or alkyl.

8. A compound according to claim 1 wherein Z is chloro, methyl, NHCOMe or OH.

9. A compound according to claim 2 wherein the phenyl group or pyridyl group is unsubstituted, or substituted by one or more substituents selected from hydroxy, halogen, methyl, hydroxymethyl and acetamido.

10. A compound according to claim 1 wherein X is selected from phenyl, 3,5-dichloro-phenyl, 3,5-dimethylphenyl, 3-hydroxyphenyl, pyridin-3-yl, pyridin-4-yl, 3-hydroxymethylphenyl and 3-acetamidophenyl.

11. A compound according to claim 1 wherein Y is an alkyl group or CONR³R⁴.

12. A compound according to claim 1 wherein R³ and R⁴ are each independently H or alkyl.

13. A compound according to claim 1 wherein Y is an ethyl group or CONMe₂.

14. A compound according to claim 1 wherein n is 1 to 4.

15. A compound according to claim 1 wherein n is 4.

16. A compound according to claim 1 wherein R¹ and R² are each independently alkyl.

17. A compound according to claim 1 wherein R¹ and R² are both methyl.

18. A pharmaceutical composition comprising a compound according to claim 1 and a pharmaceutically acceptable diluent, excipient or carrier.

19. A method of treating a muscular disorder, said method comprising administering to a subject in need thereof, a therapeutically effective amount of a compound of formula Ia, or a pharmaceutically acceptable salt thereof, 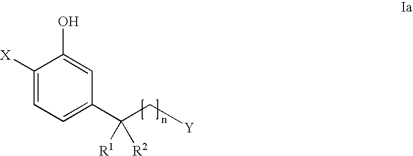
wherein
R¹ and R² are each independently H or alkyl;
Y is an alkyl group, CONR³R⁴, COOR⁵SO₂NR¹⁶R¹⁷, NHSO₂R¹⁸ or CN;
X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;
R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;
R¹² to R¹⁸ are each independently H or alkyl;
n is 1 to 6.

20. A method according to claim 19 wherein the muscular disorder is a neuromuscular disorder.

21. A method of controlling spasticity and tremors, said method comprising administering to a subject in need thereof, a therapeutically effective amount of a compound of formula Ia, or a pharmaceutically acceptable salt thereof, 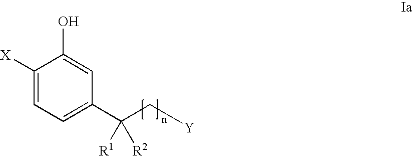
wherein
R¹ and R² are each independently H or alkyl;
Y is an alkyl group, CONR³R⁴, COOR⁵SO₂NR¹⁶R¹⁷, NHSO₂R¹⁸ or CN;
X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;
R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;
R¹² to R¹⁸ are each independently H or alkyl;
n is 1 to 6.

22. A method of treating a gastrointestinal disorder, said method comprising administering to a subject in need thereof, a therapeutically effective amount of a compound of formula Ia, or a pharmaceutically acceptable salt thereof, 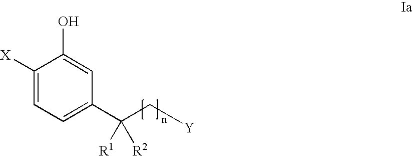
wherein
R¹ and R² are each independently H or alkyl;
Y is an alkyl group, CONR³R⁴, COOR⁵SO₂NR¹⁶R¹⁷, NHSO₂R¹⁸ or CN;
X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;
R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;
R¹² to R¹⁸ are each independently H or alkyl;
n is 1 to 6.

23. A method according to claim 22 wherein the gastrointestinal disorder is a gastric ulcer.

24. A method according to claim 22 wherein the gastrointestinal disorder is Crohn's disease.

25. A method according to claim 22 wherein the gastrointestinal disorder is secretory diarrhea.

26. A method according to claim 22 wherein the gastrointestinal disorder is paralytic ileus.

27. A method of treating neuropathic pain, said method comprising administering to a subject in need thereof, a therapeutically effective amount of a compound of formula Ia, or a pharmaceutically acceptable salt thereof, 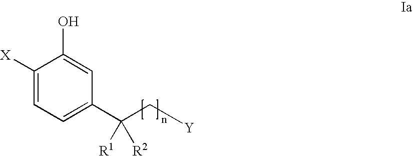
wherein
R¹ and R² are each independently H or alkyl;
Y is an alkyl group, CONR³R⁴, COOR⁵SO₂NR¹⁶R¹⁷, NHSO₂R¹⁸ or CN;
X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;
R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;
R¹² to R¹⁸ are each independently H or alkyl;
n is 1 to 6.

28. A method according to any one of claims 19, 21, 22 or 27 wherein said compound selectively modulates peripheral CB₁ receptors.

29. A method according to any one of claims 19, 21, 22 or 27, wherein said compound selectively modulates peripheral CB₁ receptors over central CB₁ receptors.

30. A method according to any one of claims 19, 21, 22 or 27, wherein the compound binds substantially exclusively to peripheral CB₁ receptors.

31. A method according to any one of claims 19, 21, 22, or 27, wherein the compound is a CB₁ receptor agonist.

32. A method according to any one of claims 19, 21, 22 or 27, wherein the compound does not substantially agonise central CB₁ receptors.

33. A method according to any one of claims 19, 21, 22 or 27, wherein the compound is substantially excluded from the CNS.

34. A method of treating a disorder associated with the modulation of peripheral CB₁ receptors, said method comprising administering to a subject in need thereof, a therapeutically effective amount of a compound of formula Ia, or a pharmaceutically acceptable salt thereof, 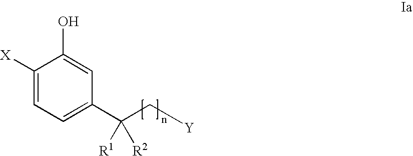
wherein
R¹ and R² are each independently H or alkyl;
Y is an alkyl group, CONR³R⁴, COOR⁵SO₂NR¹⁶R¹⁷, NHSO₂R¹⁸ or CN;
X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;
R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;
R¹² to R¹⁸ are each independently H or alkyl;
n is 1 to 6.

35. A method according to claim 34 wherein said disorder is associated with peripheral CB₁ receptor deactivation.

36. A method according to claim 34 wherein the compound does not substantially agonise central CB₁ receptors.

37. A method according to claim 34 wherein the compound binds substantially exclusively to peripheral CB₁ receptors.

38. A method according to claim 34 wherein the compound is substantially excluded from the CNS.

39. A method of inhibiting peripheral CB₁ receptors in a subject, said method comprising administering to a subject a compound of formula Ia, or a pharmaceutically acceptable salt thereof, 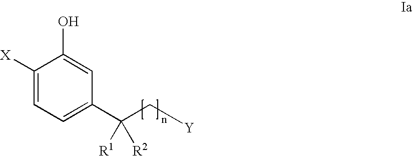
wherein
R¹ and R² are each independently H or alkyl;
Y is an alkyl group, CONR³R⁴, COOR⁵SO₂NR¹⁶R¹⁷, NHSO₂R¹⁸ or CN;
X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;
R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;
R¹² to R¹⁸ are each independently H or alkyl;
n is 1 to 6.

40. A method of modulating peripheral CB₁ receptors, said method comprising administering to a subject in need thereof, a therapeutically effective amount of a compound of formula Ia, or a pharmaceutically acceptable salt thereof, 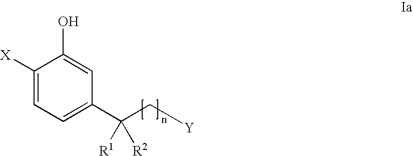
wherein
R¹ and R² are each independently H or alkyl;
Y is an alkyl group, CONR³R⁴, COOR⁵SO₂NR¹⁶R¹⁷, NHSO₂R¹⁸ or CN;
X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;
R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;
R¹² to R¹⁸ are each independently H or alkyl;
n is 1 to 6.

41. A method of identifying further compounds capable of modulating peripheral CB₁ receptors, said method comprising using in an assay a compound of formula Ia, or a pharmaceutically acceptable salt thereof, 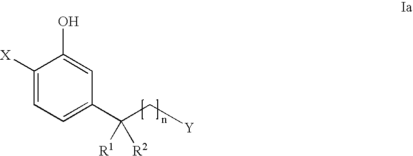
wherein
R¹ and R² are each independently H or alkyl;
Y is an alkyl group, CONR³R⁴, COOR⁵SO₂NR¹⁶R¹⁷, NHSO₂R¹⁸ or CN;
X is an aryl or heteroaryl group, each of which may be optionally substituted with one or more substituents selected from (CH₂)ₘZ where Z is halogen, OH, CN, alkyl, alkoxy, NO₂, CF₃, CONR⁶R⁷, CN, NR⁸R⁹, COOR¹⁰ or NHCOR¹¹, and m is 0 to 3;
R³ to R¹¹ are each independently H, alkyl or aryl, wherein said alkyl and aryl groups are optionally substituted by one or more substituents selected from halogen, OH, CN, alkyl, alkoxy, NO₂, CF₃, CONR¹²R¹³, CN, NH₂, COOR¹⁴, NHCOR¹⁵, and CN;
R¹² to R¹⁸ are each independently H or alkyl;
n is 1 to 6.

42. The method according to claim 41 wherein the assay is a competitive binding assay.

43. A process for the preparation of compounds of formula Ia, wherein said process comprises the steps of: 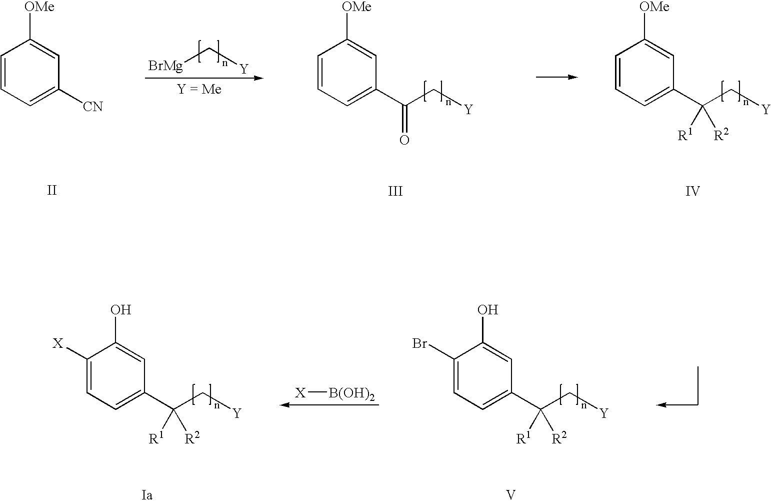
(i) reacting a compound of formula II with a compound of formula BrMg(CH₂)ₙY to form a compound of formula III;
(ii) converting said compound of formula III to a compound of formula IV;
(iii) brominating said compound of formula IV to form a compound of formula V;
(iv) converting said compound of formula V to a compound of formula Ia.

44. A process for preparing compounds of formula Ia, wherein said process comprises the steps of: 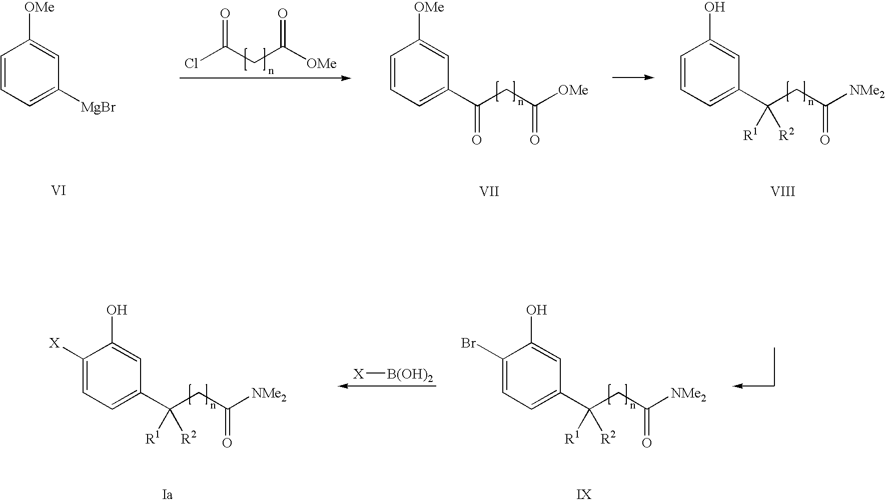
(i) reacting a compound of formula VI with a compound of formula Cl(CO)(CH₂)ₙ(CO)OMe to form a compound of formula VII;
(ii) converting said compound of formula VII to a compound of formula VIII;
(iii) brominating said compound of formula VIII to form a compound of formula IX;
(iv) converting said compound of formula IX to a compound of formula Ia.

45. The method according to any one of claims 19, 21, 22, 27, 40 or 41 wherein said compound of formula Ia is selected from: 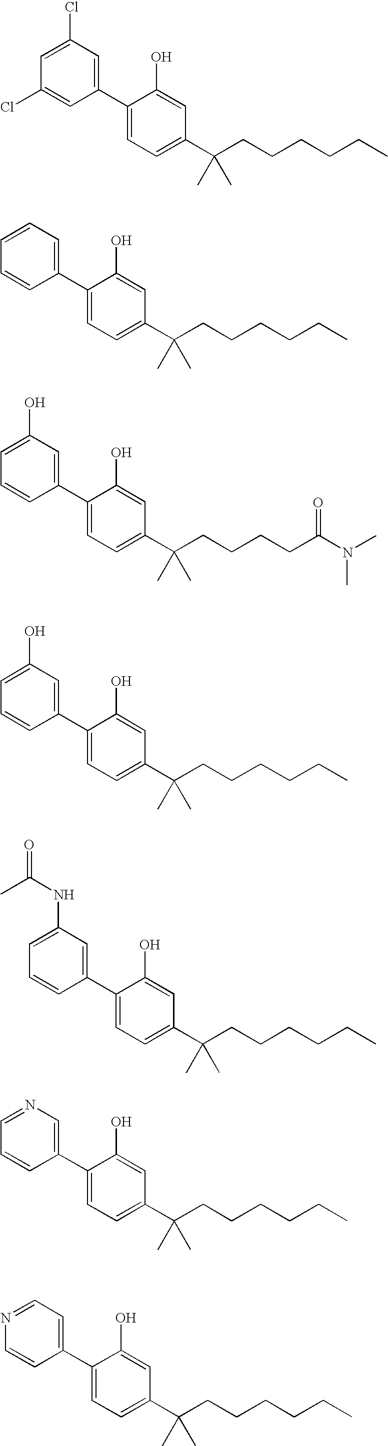 [chemical structure]
